(19) United States
(12) Patent Application Publication
(10) Pub. No.: US 2009/0023738 A1
(43) Pub. Date: Jan. 22, 2009

(54) DIAMINOPYRIMIDINES AS MODULATORS OF THE EP2 RECEPTOR

(76) Inventors: Nico Braeuer, Jena (DE); Bernd Buchmann, Hohen Neuendorf (DE); Marcus Koppitz, Berlin (DE); Antonius Ter Laak, Berlin (DE); Gernot Langer, Falkensee (DE); Bernhard Lindenthal, Berlin (DE); Olaf Peters, Tabarz (DE); Tim Wintermantel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/142,058

(22) Filed: Jun. 19, 2008

Related U.S. Application Data
(60) Provisional application No. 60/945,413, filed on Jun. 21, 2007.

(30) Foreign Application Priority Data
Jun. 21, 2007 (EP) 07075499.9

(51) Int. Cl. A61K 31/506; A61P 15/00; A61P 35/00; C07D 237/30; C07D 487/04; C07D 403/14; C07D 403/02; C07D 401/14; C07D 401/02; A61K 31/519; A61K 31/496; A61K 31/502; A61K 31/501

(52) U.S. Cl. 514/248; 544/333; 514/256; 514/252.11; 544/357; 544/237; 514/262.1; 544/262; 544/295; 514/252.19; 544/296

(57) ABSTRACT

The present invention relates to diaminopyrimidines of the general formula I, process for their preparation, and the use thereof for the manufacture of pharmaceutical compositions for the treatment of disorders and indications connected with the EP₂ receptor.

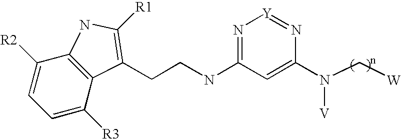

The present invention relates to diaminopyrimidines as EP₂ receptor modulators, processes for their preparation, and their use as medicaments.

It has long been known that prostaglandins are key molecules in the processes of female reproductive biology such as, for example, control of ovulation, of fertilization, of nidation, of decidualization (e.g. placenta formation) and of menstruation. Prostaglandins likewise play an important part in the pathological changes in the reproductive tract, including menorrhagia, dysmenorrhea, endometriosis and cancer. The mechanism by which prostaglandins bring about these changes has not yet been completely elucidated. Recent results indicate that prostaglandins, their receptors and signal transduction pathways thereof are involved in processes such as angiogenesis, apoptosis, proliferation, and in inflammatory/antiinflammatory and immunological processes.

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP₁, EP₂, EP₃ and EP₄ receptors. The receptor subtypes to which prostaglandin E₂ binds appear to be of particular interest for the receptor-mediated effects which are involved in the control of fertility. It has thus been possible to show that the reproductive functions in EP₂ knockout mice (EP₂^{-/-}), i.e. in mice no longer having a functional PGE₂ receptor of the EP₂ subtype, are impaired, and that these animals have a smaller “litter size” (Matsumoto et al., 2001, Biology of Reproduction 64, 1557-1565). It was likewise possible to show that these EP₂ knockout mice (Hizaki et al. Proc Natl Acad Sci U.S.A. 1999 Aug. 31; 96(18):10501-10506) show distinctly reduced cumulus expansion and severe subfertility, which is to be regarded as causally connected with diminished reproductive processes such as ovulation and fertilization.

The EP₂ receptor accordingly represents an important target for developing medicaments for controlling female fertility. The existence of the 4 subclasses of the PGE₂ receptor opens up the possibility of targeted development of selectively active compounds. However, to date, scarcely any selective EP₂ receptor ligands which bind to the EP₂ subtypes of the PGE₂ receptor are known, since most known compounds also bind to the other PGE₂ receptor subtypes such as, for example, to the EP₄ receptor.

EP₂ receptor antagonists are described, for example in the application US2005059742 (Jabbour, Medical Research Concil). A method in which an EP₂ and/or an EP₄ antagonist can be employed for the treatment of menorrhagia and dysmenorrhea is claimed. AH6809 is disclosed as antagonist of the EP₂ or EP₄ receptor, but no other specific antagonists and no new compounds are disclosed.

In an earlier application of the same group (EP1467738), EP₂ or EP₄ antagonists are described for the treatment of pathological conditions such as, for example, allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

Ono Pharmaceutical claims in the application WO03/016254 the preparation of benzene or saturated carboxylic acid derivatives which are substituted by aryl or heterocycles, inter alia as PGE₂ receptor antagonists. The disclosed compounds are claimed for the treatment of a large number of disorders, including allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

The application WO04/39807 of Merck Frosst, Canada, discloses the preparation of pyridopyrrolizines and pyridoindolizines. However, these compounds are distinguished by good binding to the PGD₂ receptor, and this receptor represents a different subtype of the prostaglandin receptor.

Naphthalene derivatives as EP₄ receptor ligands are disclosed in application US2004102508 of SmithKline Beecham Corporation. The claimed compounds are used for the treatment or prophylaxis of pain, allergic reactions and neurodegenerative disorders.

EP₄ antagonists (γ-lactams) are claimed in the application WO03/103604 (Applied Research Systems). The compounds bind approximately 60-fold better to the EP₄ than to the EP₂ receptor and are claimed inter alia for the treatment of premature labor, dysmenorrhea, asthma, infertility or fertility impairments. The same company claims in the applications WO03/053923 (substituted pyrrolidines) or WO03/035064 (substituted pyrazolidinones) compounds for the treatment of disorders associated with prostaglandins, such as, for example, infertility, hypertension and osteoporosis. The compounds bind to the EP₄₋ and to the EP₂ receptor subtypes. The application WO03/037433 claims ω-cycloalkyl, 17 heteroaryl prostaglandin derivatives as EP₂ receptor antagonists, in particular for the treatment of elevated intraocular pressure.

The application WO03/064391 (Pfizer Products) describes metabolites of [3-[[N-(4-tert-butylbenzyl)(pyridin-3-ylsulfonyl)amino]methyl]acetic acid which inhibit the binding of [³H] prostaglandin E₂ to the EP₂ receptor. The use of these metabolites for the treatment of osteoporosis is disclosed.

Tani et al. claim in the application US2005124577 8-azaprostaglandin derivatives for the treatment of immunological disorders, allergic disorders, premature labor, abortion, etc. The compounds bind to the EP₂ and to the EP₄ receptor.

European patent application EP 1306087 describes EP₂ receptor agonists which are used for the treatment of erectile dysfunction (Ono Pharmaceuticals). The same class of structures is described in European patent EP 860430 (Ono Pharmaceuticals), and their use for the manufacture of a medicament for the treatment of immunological disorders, asthma and abortion is claimed. WO04/009117 describes EP₂ and EP₄ receptor agonists for the treatment of disorders caused by uterine contraction, for example painful menstruation (Ono Pharmaceuticals).

The applications WO03/74483 and WO03/09872 describe agonists which bind equally to the EP₂ and to the EP₄ receptor (Ono Pharmaceuticals).

Agonists of the EP₂ and of the EP₄ receptors are frequently described in connection with the treatment of osteoporosis (WO99/19300 (Pfizer), US2003/0166631 (Dumont Francis), WO03/77910 (Pfizer), WO03/45371 (Pfizer), WO03/74483 and WO03/09872 (Ono Pharmaceuticals)) and for glaucoma treatment (WO04/37813, WO04/37786, WO04/19938, WO03/103772, WO03/103664, WO03/40123, WO03/47513, WO03/47417 (Merck Frosst Canada)) and U.S. Pat. No. 6,410,591 and U.S. Pat. No. 6,747,037 (Allergan).

The patent application WO04/12656 (Applied Research Systems) claims EP₂ receptor agonists in connection with inflammation.

The patent application WO03/77919 (Merck & Co. Inc.) claims EP₄ receptor agonists for the treatment of fertility.

However, to date, no selective EP₂ receptor agonists and antagonists which control the processes which are ultimately responsible for ovulation, fertilization, nidation and decidualization and thus contribute to promoting or inhibiting fertility are known.

It is therefore an object of the present invention to provide stable EP₂ receptor antagonists.

This object is achieved by the provision of compounds of the general formula I

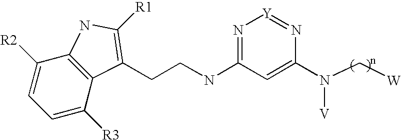

where
Y is a CH group or a C(C₁-C₄-alkyl) group,
V is a hydrogen, a C₁-C₄-alkyl group,
n is 0, 1 or 2,
W is a 6-10-membered, mono- or bicyclic aryl ring which is in each case unsubstituted or optionally substituted once to three times, a 5-10-membered, mono- or bicyclic heteroaryl ring which is in each case unsubstituted or optionally substituted once to three times,
   an 8-12-membered aryl- or heteroaryl-cycloalkyl or -cycloalkenyl group which is in each case unsubstituted or optionally substituted once to three times,
   an 8-12-membered aryl- or heteroaryl-heterocyclyl or -heterocyclenyl group which is in each case unsubstituted or optionally substituted once to three times,
   a 3-12-membered, mono-, bi- or tricyclic cycloalkyl radical which is in each case unsubstituted or optionally substituted once,
      where the substituents are linked either directly or via a spacer U to W and may be selected from the group of halogen, cyano, R⁴, OR⁴, OC(O)R⁴, S(O)NR⁴ where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
   or
   in the case where n=0 together with V is a pyrrolidine, piperidine, morpholine or thiomorpholine residue which is in each case unsubstituted or optionally substituted once, or else
   in the case where n=0 together with V is a piperazine residue, which is unsubstituted or optionally N-substituted,
      where the substituents are linked either directly or via a spacer U to W and may be selected from the group of halogen, cyano, R⁴, OR⁴, OC(O)R⁴, S(O)ₙR⁴ where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
U is a C₁-C₄-alkylene, C₂-C₄-alkenylidene, C₂-C₄-alkynylidene, O—C₁-C₄-alkylene, C(O)—C₁-C₄-alkylene, S(O)ₙ—C₁-C₄-alkylene, where n is 0, 1, 2, N(R⁵)—C₁-C₄-alkylene, C(O)—N(R⁵)—C₁-C₄-alkylene, N(R⁵)—C(O)—C₁-C₄-alkylene spacer,
R¹ is a C₁-C₄-alkyl group or cyano,
R² is a hydrogen, halogen, cyano, a C₁-C₄-alkyl group,
R³ is a hydrogen, halogen, cyano, a C₁-C₄-alkyl group,
R⁴ is a hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or heteroaryl group, where the aryl radical is 6-membered and the heteroaryl radical is 5 or 6-membered,
R⁵ is a hydrogen, a C₁-C₄-alkyl group and
R⁴, R⁵ together form a 3-6-membered cycloalkyl or a heteroatom-containing ring,
   and the isomers, diastereomers, enantiomers and salts thereof, and cyclodextrin clathrates, which overcome the known disadvantages and have improved properties, i.e. a good activity, good solubility and stability.

Examples of the C₁-C₄-alkyl substituents indicated under V, Y, R¹, R², R³, R⁴ and R⁵ are a methyl, ethyl, n-propyl, n-butyl group, and of the branched C₃-C₄-alkyl groups are an isopropyl, isobutyl, sec-butyl, tert-butyl group. The alkyl groups may optionally be substituted once or more than once by halogen atoms (e.g. fluorine, chlorine or bromine).

The C₂-C₄-alkenyl substituents in R⁴ are in each case straight-chain or branched, meaning for example the following radicals: vinyl-, allyl-, homoallyl-, (E)-but-2-enyl-, (Z)-but-2-enyl-, 2-methylvinyl-.

The alkenyl groups may optionally be substituted once or more than once by halogen atoms (e.g. fluorine, chlorine or bromine).

The C₂-C₄-alkynyl substituents R⁴ are in each case straight-chain or branched, meaning for example the following radicals: ethynyl, prop-1-ynyl, but-1-ynyl, but-2-ynyl.

The alkynyl groups may optionally be substituted once by halogen atoms (e.g. fluorine, chlorine or bromine).

The C₁-C₄-alkylene spacers indicated under U are straight-chain or branched spacers, for example methylene, ethylene, propylene, butylene spacers.

The C₁-C₄-alkylene spacers may optionally be substituted once or more than once by halogen atoms, (e.g. fluorine, chlorine or bromine).

The C₂-C₄-alkenylidene spacers in U are in each case straight-chain or branched, meaning for example the following radicals: ethenylidene, propenylidene, butenylidene.

The C₂-C₄-alkenylidene groups may be substituted once or more than once by halogen atoms (e.g. fluorine, chlorine or bromine).

The C₂-C₄-alkynylidene spacers in U are in each case straight-chain or branched, meaning for example the following radicals: ethynylidene, propynylidene, butynylidene.

The C₂-C₄-alkynylidene groups may optionally be substituted once by halogen atoms (e.g. fluorine, chlorine or bromine).

Halogen means the following: fluorine, chlorine, bromine, iodine.

The C₃-C₁₂-cycloalkyl indicated under W takes the form of monocyclic alkyl rings such as cyclopropyl, cyclobutyl, cyclopentyl, cyclohexyl or cycloheptyl, or cyclooctyl, but also bicyclic rings such as, for example, decahydronaphthalene, tricyclic rings or bridged rings such as, for example, adamantanyl, and heteroatom-containing heterocycles such as, for example, aziridinyl, azetidinyl, pyrrolidinyl, piperidinyl, piperazinyl, morpholinyl, azepanyl, [1,4]-diazepanyl, tetrahydrofuranyl, thiomorpholinyl.

The C₃-C₁₂-cycloalkyl groups are linked via one of the substitutable positions and may optionally be substituted once to twice by halogen atoms, (e.g. fluorine, chlorine or bromine) or an oxo group. The N and S atoms may optionally be oxidized to an N-oxide, S-oxide, S,S-dioxide.

The C₃-C₆-cycloalkyl indicated under R⁴ takes the form of alkyl rings such as cyclopropyl, cyclobutyl, cyclopentyl or cyclohexyl, and of heteroatom-containing heterocycles such as, for example, aziridinyl, azetidinyl, pyrrolidinyl, tetrahydrofuranyl, piperidinyl, piperazinyl, morpholinyl, thiomorpholinyl.

The C₃-C₆-cycloalkyl groups are linked via one of the substitutable positions and may optionally be substituted once to twice by halogen atoms (e.g. fluorine, chlorine or bromine) or an oxo group. The N and S atoms may optionally be oxidized to an N-oxide, S-oxide, S,S-dioxide.

The 6-10-membered, mono- or bicyclic aryl radical which may optionally be substituted once to three times and which is indicated in W is connected to the framework via one of the possible linkage positions. The 6-10-membered, mono- or bicyclic aryl or heteroaryl radical may optionally be substituted once to three times by halogen atoms (e.g. fluorine, chlorine or bromine), C₁-C₄-alkyl groups or a hydroxy group.

Examples which may be mentioned for a 6-10-membered, mono- or bicyclic aryl radical are the following: phenyl, naphthyl.

The 5-10-membered, mono- or bicyclic heteroaryl radical which may optionally be substituted once to three times and which is indicated in W means 5-10-membered ring systems which may, instead of the carbon, comprise one or more, identical or different heteroatoms such as oxygen, nitrogen or sulfur in the ring, may be mono- or bicyclic and are connected to the framework via one of the possible linkage positions. The 5-10-membered, mono- or bicyclic heteroaryl radicals may optionally be substituted once to three times by halogen atoms (e.g fluorine, chlorine or bromine), C₁-C₄-alkyl groups or a hydroxy group. If the heteroaryl radical is substituted by a hydroxy group, the corresponding tautomers are included if the hydroxy group on the heteroaryl radical is capable thereof. The N atoms may optionally be oxidized to an N-oxide.

The 5-10-membered, mono- or bicyclic heteroaryl radicals may take the form of a pyridyl, pyrimidyl, quinolinyl, isoquinolinyl, phthalazinyl, quinazolinyl, quinoxalinyl, cinnolinyl, benzofuranyl, benzothienyl, indolyl, benzimidazolyl, 2,1,3-benzothiadiazolyl, 1H-benzotriazolyl, benzothiazolyl, benzoxazolyl, benzisoxazolyl, furanyl, thienyl, oxazolyl, isoxazolyl, isothiazolyl, thiazolyl, isothiazolyl, pyrrolyl, pyrazolyl, pyrazinyl, pyridazinyl, triazinyl, carbazolyl, 1H-pyrazolo[3,4-d]pyrimidyl, 1H-indazolyl, triazolyl, oxadiazolyl, tetrazolyl or an imidazolyl group which is linked via one of the substitutable positions.

The 6-membered aryl radical indicated in R⁴ is a phenyl radical which may optionally be substituted once to twice by halogen atoms (e.g. fluorine, chlorine or bromine), C₁-C₄-alkyl groups or a hydroxy group.

The 5-6-membered heteroaryl radical indicated in R⁴ means 5-6-membered ring systems which, instead of the carbon, may comprise one or more, identical or different heteroatoms such as oxygen, nitrogen or sulfur in the ring, and are connected to the framework via one of the possible linkage positions. The 5-6-membered heteroaryl radicals may optionally be substituted once to twice by halogen atoms (e.g. fluorine, chlorine or bromine), C₁-C₄-alkyl groups or a hydroxy group. If the heteroaryl radical is substituted by a hydroxy group, the corresponding tautomers are included if the hydroxy group on the heteroaryl radical is capable thereof. The N atoms may optionally be oxidized to an N-oxide.

The 5-6-membered heteroaryl groups may take the form of a pyridyl, pyrimidyl, furanyl, thienyl, oxazolyl, isoxazolyl, thiazolyl, isothiazolyl, pyrrolyl, pyrazolyl, pyrazinyl, pyridazinyl, triazinyl, triazolyl, oxadiazolyl, tetrazolyl or an imidazolyl group which is linked via one of the substitutable positions.

The 8-12-membered aryl- or heteroaryl-cycloalkyl or -cycloalkenyl groups mentioned under W are unsubstituted or optionally substituted once to three times and comprise optionally instead of the carbon one or more, identical or different heteroatoms such as oxygen, nitrogen or sulfur in the heteroaryl moiety. The nitrogen atoms are optionally oxidized to an N-oxide. The 8-12-membered aryl- or heteroaryl-cycloalkyl or -cycloalkenyl groups are linked via one of the substitutable positions and additionally substituted optionally in the cycloalkyl or cycloalkenyl moiety once to twice by an oxo group. The 8-12-membered aryl- or heteroaryl-cycloalkyl or -cycloalkenyl groups may optionally be substituted once to three times by halogen atoms (e.g. fluorine, chlorine or bromine) or C₁-C₄-alkyl groups.

An aryl-cycloalkyl group is for example 1,2,3,4-tetrahydronaphthalenyl, indanyl, 3,4-dihydro-2H-naphthalen-1-onyl, indan-1-onyl.

A heteroaryl-cycloalkyl group is for example 5,6,7,8-tetrahydroquinolinyl, 5,6,7,8-tetrahydroisoquinolinyl, 5,6,7,8-tetrahydroquinazolinyl, 5,6,7,8-tetrahydroquinoxalinyl, 4,5,6,7-tetrahydro-1H-benzimidazolyl, 4,5,6,7-tetrahydro-benzoxazolyl, 4,5,6,7-tetrahydrobenzthiazolyl, 2,4,5,6-tetrahydrocyclopenta-pyrazolyl.

An aryl-cycloalkenyl group is for example 1,2-dihydronaphthalenyl, 1H-indenyl.

A hetaryl-cycloalkenyl group is for example 5,6-dihydroquinolinyl, 5,6-dihydroisoquinolinyl, 5,6-dihydroquinazolinyl, 5,6-dihydroquinoxalinyl, 4,5-dihydro-1H-benzimidazolyl, 4,5-dihydrobenzoxazolyl, 4,5-dihydro-benzthiazolyl.

The 8-12-membered aryl- or heteroaryl-heterocyclyl or -heterocyclenyl groups mentioned under W are unsubstituted or optionally substituted once to three times and comprise one or more, identical or different heteroatoms such as oxygen, nitrogen or sulfur in the heteroaryl and heterocyclyl or heterocyclenyl moiety. The nitrogen atoms in the heteroaryl moiety are optionally oxidized to an N-oxide. The oxygen, nitrogen or sulfur atoms in the heterocyclyl or heterocyclenyl moiety are optionally oxidized to an N-oxide, S-oxide, S,S-dioxide. The 8-12-membered aryl- or heteroaryl-heterocyclyl or -heterocyclenyl groups are linked via one of the substitutable positions and additionally are optionally substituted in the heterocyclyl or heterocyclenyl moiety once to twice by an oxo group. The 8-12-membered aryl- or heteroaryl-heterocyclyl or -heterocyclenyl groups may optionally be substituted once to three times by halogen atoms (e.g. fluorine, chlorine or bromine) or C₁-C₄-alkyl groups.

An aryl-heterocyclyl group is for example 1,2,3,4-tetrahydroquinolinyl, 1,2,3,4-tetrahydroisoquinolinyl, 1,2,3,4-tetrahydroquinazolinyl, 1,2,3,4-tetrahydro-quinoxalinyl, 1,2,3,4-tetrahydrophthalazinyl, 2,3-dihydro-1H-indolyl, 2,3-dihydro-benzofuranyl, 2,3-dihydro-1H-isoindolyl, benzo[1,3]dioxolyl, 2,3-dihydro-benzoxazolyl, chromanyl, 2,3-dihydrobenzo[1,4]dioxinyl, 2,3-dihydrophthalazine-1,4-dionyl, isoindole-1,3-dionyl, 2-methylisoindole-1,3-dionyl, 2,3-dihydro-isoindol-1-onyl.

A heteroaryl-heterocyclyl group is for example 2,3-dihydro-1H-pyrrol-[3,4-b]quinolin-2-yl, 1,2,3,4-tetrahydrobenz[b][1,7]naphthyridin-2-yl, 1,2,3,4-tetrahydrobenz[b][1,6]naphthyridin-2-yl, 1,2,3,4-tetrahydro-9H-pyrido[3,4-b]indol-2-yl, 1,2,3,4-tetrahydro-9H-pyrido[4,3-b]indol-2-yl, 2,3-dihydro-1H-pyrrolo[3,4-b]indol-2-yl, 1H-2,3,4,5-tetrahydroazepino[3,4-b]indol-2-yl, 1H-2,3,4,5-tetrahydroazepino[4,3-b]indol-3-yl, 1H-2,3,4,5-tetrahydro-azepino[4,5-b]indol-2-yl, 5,6,7,8-tetrahydro[1,7]naphthyridyl, 1,2,3,4-tetrahydro[2,7]naphthyridyl, 2,3-dihydro[1,4]dioxino[2,3-b]pyridyl, 2,3-dihydro-[1,4]dioxino[2,3-b]pyridyl, 3,4-dihydro-2H-1-oxa[4,6]diazanaphthalenyl, 4,5,6,7-tetrahydro-3H-imidazo[4,5-c]pyridyl, 6,7-dihydro[5,8]diazanaphthalenyl, 1,2,3,4-tetrahydro[1,5]-naphthyridinyl, 1,2,3,4-tetrahydro[1,6]naphthyridinyl, 1,2,3,4-tetrahydro[1,7]naphthyridinyl, 1,2,3,4-tetrahydro[1,8]naphthyridinyl, 1,2,3,4-tetra-hydro[2,6]naphthyridinyl.

An aryl-heterocyclenyl group is for example 3H-indolinyl, 1H-2-oxoquinolyl, 2H-1-oxoisoquinolyl, 1,2-dihydroquinolinyl, 3,4-dihydroquinolinyl, 1,2-dihydroisoquinolinyl, 3,4-dihydroisoquinolinyl, 4H-chromenyl, 4-methyl-chromen-2-onyl.

A heteroaryl-heterocyclenyl group is for example 7,8-dihydro[1,7]naphthyridinyl, 1,2-dihydro[2,7]-naphthyridinyl, 6,7-dihydro-3H-imidazo[4,5-c]pyridyl, 1,2-dihydrol,5-naphthyridinyl, 1,2-dihydro-1,6-naphthyridinyl, 1,2-dihydro-1,7-naphthyridinyl, 1,2-dihydro-1,8-naphthyridinyl, 1,2-dihydro-2,6-naphthyridinyl.

The 3-6-membered cycloalkyl ring formed by ring closure of R⁴ and R⁵ may be for example a cyclopropyl, cyclobutyl, cyclopentyl or cyclohexyl.

Examples of a 3-6-membered, heteroatom-containing ring formed by ring closure of R⁴ and R⁵ which may be mentioned are the following: aziridinyl, azetidinyl, pyrrolidinyl, piperidinyl, piperazinyl, morpholinyl, thiomorpholinyl. The N and S atoms may optionally be oxidized to an N-oxide, S-oxide, S,S-dioxide.

Preference is given to the compounds of the general formula I, where
Y is a CH group or a C(C₁-C₄-alkyl) group,
V is a hydrogen, a CH₃ group,
n is 0, 1 or 2,
W is a 6-10-membered, mono- or bicyclic aryl ring which is in each case unsubstituted or optionally substituted once to three times, a 5-10-membered, mono- or bicyclic heteroaryl ring which is in each case unsubstituted or optionally substituted once to three times,
   an 8-12-membered aryl- or heteroaryl-cycloalkyl or -cycloalkenyl group which is in each case unsubstituted or optionally substituted once to three times,
   an 8-12-membered aryl- or heteroaryl-heterocyclyl or -heterocyclenyl group which is in each case unsubstituted or optionally substituted once to three times,
   a 3-6 membered cycloalkyl radical which is in each case unsubstituted or optionally substituted once,
      where the substituents are linked either directly or via a spacer U to W and may be selected from the group of halogen, cyano, R⁴, OR⁴, OC(O)R⁴, S(O)NR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
   or
   in the case where n=0 together with V is a pyrrolidine, piperidine, morpholine or thiomorpholine residue which is in each case unsubstituted or optionally substituted once,
   or else
   in the case where n=0 together with V is a piperazine radical, which is unsubstituted or optionally N-substituted,
where the substituents are linked either directly or via a spacer U to W and may be selected from the group of halogen, cyano, R⁴, OR⁴, OC(O)R⁴, S(O)NR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
U is a C₁-C₄-alkylene, C₂-C₄-alkenylidene, C₂-C₄-alkynylidene, O—C₁-C₄-alkylene, C(O)—C₁-C₄-alkylene, S(O)ₙ—C₁-C₄-alkylene, where n is 0, 1, 2, N(R⁵)—C₁-C₄-alkylene, C(O)—N(R⁵)—C₁-C₄-alkylene, N(R⁵)—C(O)—C₁-C₄-alkylene spacer,
R¹ is a C₁-C₄-alkyl group or cyano,
R² is a hydrogen, halogen, cyano, a C₁-C₄-alkyl group,
R³ is a hydrogen, halogen, cyano, a C₁-C₄-alkyl group,
R⁴ is a hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or heteroaryl group, where the aryl radical is 6-membered and the heteroaryl radical is 5 or 6-membered,
R⁵ is a hydrogen, a C₁-C₄-alkyl group,
R⁴, R⁵ together form a 3-6-membered cycloalkyl or a heteroatom-containing ring.

Preference is given to the compounds of the general formula I, where
Y is a CH group or a C(C₁-alkyl) group,
V is a hydrogen, a CH₃ group,
n is 0, 1 or 2,
W is a 6-10-membered, mono- or bicyclic aryl ring which is in each case unsubstituted or optionally substituted once to three times, a 5-10-membered, mono- or bicyclic heteroaryl ring which is in each case unsubstituted or optionally substituted once to three times,
   an 8-12-membered aryl- or heteroaryl-cycloalkyl or -cycloalkenyl group which is in each case unsubstituted or optionally substituted once to three times,
   an 8-12-membered aryl- or heteroaryl-heterocyclyl or -heterocyclenyl group which is in each case unsubstituted or optionally substituted once to three times,
   a 3-6-membered cycloalkyl radical which is in each case unsubstituted or optionally substituted once,
      where the substituents are linked either directly or via a spacer U to W and may be selected from the group of halogen, cyano, R⁴, OR⁴, OC(O)R⁴, S(O)NR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
   or
   in the case where n=0 together with V is a pyrrolidine, piperidine, morpholine or thiomorpholine residue which is in each case unsubstituted or optionally substituted once, or else
   in the case where n=0 together with V is a piperazine residue which is unsubstituted or optionally N-substituted,
      where the substituents are linked either directly or via a spacer U to W and may be selected from the group of halogen, cyano, R⁴, OR⁴, OC(O)R⁴, S(O)ₙR⁴ where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
U is a C₁-C₄-alkylene, C₂-C₄-alkenylidene, C₂-C₄-alkynylidene, O—C₁-C₄-alkylene, C(O)—C₁-C₄-alkylene, S(O)ₙ—C₁-C₄-alkylene, where n is 0, 1, 2, N(R⁵)—C₁-C₄-alkylene, C(O)—N(R⁵)—C₁-C₄-alkylene, N(R⁵)—C(O)—C₁-C₄-alkylene spacer,
R¹ is a C₁₋alkyl group or cyano,
R² is a hydrogen, halogen, cyano, a C₁-alkyl group,
R³ is a hydrogen, halogen, cyano, a C₁-alkyl group,
R⁴ is a hydrogen, a C₁-C₄-alkyl group, a C₂-C₄-alkenyl group, a C₂-C₄-alkynyl group, a C₃-C₆— cycloalkyl group, a CH₂—C₃-C₆-cycloalkyl group, a 6-membered aryl ring, a 5-6-membered heteroaryl ring or a CH₂-aryl or heteroaryl group, where the aryl radical is 6-membered and the heteroaryl radical is 5 or 6-membered,
R⁵ is a hydrogen, a C₁-C₄-alkyl group and
R⁴, R⁵ together form a 3-6-membered cycloalkyl or a heteroatom-containing ring.

The following compounds corresponding to the present invention are very particularly preferred:
1. N-[2-(7-Fluoro-2,4-dimethyl-1H-indol-3-yl)ethyl]-N′-pyridin-2-ylpyrimidine-4,6-diamine
2. N-[2-(7-Fluoro-2,4-dimethyl-1H-indol-3-yl)ethyl]-N′-pyridin-3-ylpyrimidine-4,6-diamine
3. N-(3-Chlorophenyl)-N′-[2-(7-fluoro-2,4-dimethyl-1H-indol-3-yl)ethyl]-pyrimidine-4,6-diamine
4. N-[2-(7-Fluoro-2,4-dimethyl-1H-indol-3-yl)ethyl]-N′-(3-trifluoromethylphenyl)-pyrimidine-4,6-diamine
5. N-[2-(7-Fluoro-2,4-dimethyl-1H-indol-3-yl)ethyl]-N′-(4-fluorophenyl)-pyrimidine-4,6-diamine
6. N-[2-(7-Fluoro-2,4-dimethyl-1H-indol-3-yl)ethyl]-N′-pyridin-3-ylmethylpyrimidine-4,6-diamine
7. N-[2-(7-Fluoro-2,4-dimethyl-1H-indol-3-yl)ethyl]-N′-phenylpyrimidine-4,6-diamine
8. N-[2-(7-Fluoro-2,4-dimethyl-1H-indol-3-yl)ethyl]-N′-(2-methoxyphenyl)-pyrimidine-4,6-diamine
9. N-[2-(7-Fluoro-2,4-dimethyl-1H-indol-3-yl)ethyl]-N′-(3-methoxyphenyl)-pyrimidine-4,6-diamine
10. N-[2-(7-Fluoro-2,4-dimethyl-1H-indol-3-yl)ethyl]-N′-(4-methoxyphenyl)-pyrimidine-4,6-diamine
11. N-(4-Chlorophenyl)-N′-[2-(7-fluoro-2,4-dimethyl-1H-indol-3-yl)ethyl]-pyrimidine-4,6-diamine
12. N-Cyclohexyl-N′-[2-(7-fluoro-2,4-dimethyl-1H-indol-3-yl)ethyl]pyrimidine-4,6-diamine
13. N-(4-Dimethylaminophenyl)-N′-[2-(7-fluoro-2,4-dimethyl-1H-indol-3-yl)ethyl]-pyrimidine-4,6-diamine
14. N-[2-(7-Fluoro-2,4-dimethyl-1H-indol-3-yl)ethyl]-N′-pyrazin-2-ylpyrimidine-4,6-diamine
15. N-Benzyl-N′-[2-(7-fluoro-2,4-dimethyl-1H-indol-3-yl)ethyl]pyrimidine-4,6-diamine
16. N-[2-(7-Fluoro-2,4-dimethyl-1H-indol-3-yl)ethyl]-N′-(4-methoxybenzyl)-pyrimidine-4,6-diamine
17. N-Biphenyl-2-ylmethyl-N′-[2-(7-fluoro-2,4-dimethyl-1H-indol-3-yl)ethyl]-pyrimidine-4,6-diamine
18. N-[2-(7-Fluoro-2,4-dimethyl-1H-indol-3-yl)ethyl]-N′-(4-[1,2,4]triazol-1-yl-phenyl)pyrimidine-4,6-diamine
19. [2-(7-Fluoro-2,4-dimethyl-1H-indol-3-yl)ethyl]-[6-(2,3,5,6-tetrahydro-[1,2′]bipyrazinyl-4-yl)pyrimidin-4-yl]amine
20. N-[2-(7-Fluoro-2,4-dimethyl-1H-indol-3-yl)ethyl]-N′-(4-methylbenzyl)-pyrimidine-4,6-diamine
21. N-[2-(7-Fluoro-2,4-dimethyl-1H-indol-3-yl)ethyl]-N′-(4-trifluoromethylphenyl)-pyrimidine-4,6-diamine
22. N-Biphenyl-3-ylmethyl-N′-[2-(7-fluoro-2,4-dimethyl-1H-indol-3-yl)ethyl]-pyrimidine-4,6-diamine
23. 4-{6-[2-(7-Fluoro-2,4-dimethyl-1H-indol-3-yl)ethylamino]pyrimidin-4-ylamino}-N-thiazol-2-ylbenzenesulfonamide
24. N-(4,6-Dimethylpyrimidin-2-yl)-4-{6-[2-(7-fluoro-2,4-dimethyl-1H-indol-3-yl)ethylamino]pyrimidin-4-ylamino}benzenesulfonamide
25. N-[2-(7-Fluoro-2,4-dimethyl-1H-indol-3-yl)ethyl]-N′-(6-methylpyridin-2-yl)-pyrimidine-4,6-diamine
26. 5-{6-[2-(7-Fluoro-2,4-dimethyl-1H-indol-3-yl)ethylamino]pyrimidin-4-ylamino}-indan-1-one
27. 6-{6-[2-(7-Fluoro-2,4-dimethyl-1H-indol-3-yl)ethylamino]pyrimidin-4-ylamino}-3,4-dihydro-2H-naphthalen-1-one
28. 5-{6-[2-(7-Fluoro-2,4-dimethyl-1H-indol-3-yl)ethylamino]pyrimidin-4-ylamino}-isoindole-1,3-dione
29. 6-{6-[2-(7-Fluoro-2,4-dimethyl-1H-indol-3-yl)ethylamino]pyrimidin-4-ylamino}-nicotinamide
30. N-[2-(7-Fluoro-2,4-dimethyl-1H-indol-3-yl)ethyl]-N′-naphthalen-1-yl-pyrimidine-4,6-diamine
31. N-Benzo[1,3]dioxol-5-yl-N′-[2-(7-fluoro-2,4-dimethyl-1H-indol-3-yl)ethyl]-pyrimidine-4,6-diamine
32. N-[2-(7-Fluoro-2,4-dimethyl-1H-indol-3-yl)ethyl]-N′-(1H-indol-5-yl)-pyrimidine-4,6-diamine
33. N-(1H-Benzotriazol-5-yl)-N′-[2-(7-fluoro-2,4-dimethyl-1H-indol-3-yl)ethyl]-pyrimidine-4,6-diamine
34. N-[2-(7-Fluoro-2,4-dimethyl-1H-indol-3-yl)ethyl]-N′-indan-5-ylpyrimidine-4,6-diamine
35. 4-{6-[2-(7-Fluoro-2,4-dimethyl-1H-indol-3-yl)ethylamino]pyrimidin-4-ylamino}-isoindole-1,3-dione
36. 4-{6-[2-(7-Fluoro-2,4-dimethyl-1H-indol-3-yl)ethylamino]pyrimidin-4-ylamino}-benzamide
37. 6-{6-[2-(7-Fluoro-2,4-dimethyl-1H-indol-3-yl)ethylamino]pyrimidin-4-ylamino}-2,3-dihydrophthalazine-1,4-dione
38. N-[2-(7-Fluoro-2,4-dimethyl-1H-indol-3-yl)ethyl]-N′-(5-methyl-2H-pyrazol-3-yl)pyrimidine-4,6-diamine
39. N-[2-(7-Fluoro-2,4-dimethyl-1H-indol-3-yl)ethyl]-N′-quinolin-3-ylpyrimidine-4,6-diamine
40. N-[2-(7-Fluoro-2,4-dimethyl-1H-indol-3-yl)ethyl]-N′-quinolin-5-ylpyrimidine-4,6-diamine
41. N-[2-(7-Fluoro-2,4-dimethyl-1H-indol-3-yl)ethyl]-N′-quinolin-8-ylpyrimidine-4,6-diamine
42. 5-{6-[2-(7-Fluoro-2,4-dimethyl-1H-indol-3-yl)ethylamino]pyrimidin-4-ylamino}-2-methylisoindole-1,3-dione
43. N-[2-(7-Fluoro-2,4-dimethyl-1H-indol-3-yl)ethyl]-N′-(1H-pyrazolo[3,4-d]pyrimidin-4-yl)pyrimidine-4,6-diamine
44. N-(2,5-Dimethyl-2H-pyrazol-3-yl)-N′-[2-(7-fluoro-2,4-dimethyl-1H-indol-3-yl)ethyl]pyrimidine-4,6-diamine
45. N-[2-(7-Fluoro-2,4-dimethyl-1H-indol-3-yl)ethyl]-N′-(2-trifluoromethyl-1H-benzoimidazol-5-yl)pyrimidine-4,6-diamine
46. N-[2-(7-Fluoro-2,4-dimethyl-1H-indol-3-yl)ethyl]-N′-[3-(1H-tetrazol-5-yl)-phenyl]pyrimidine-4,6-diamine
47. 3-{6-[2-(7-Fluoro-2,4-dimethyl-1H-indol-3-yl)ethylamino]pyrimidin-4-ylamino}-benzenesulfonamide
48. N-[2-(7-Fluoro-2,4-dimethyl-1H-indol-3-yl)ethyl]-N′-(1H-indazol-5-yl)-pyrimidine-4,6-diamine
49. N-[2-(7-Fluoro-2,4-dimethyl-1H-indol-3-yl)ethyl]-N′-(1H-indazol-6-yl)-pyrimidine-4,6-diamine
50. N-[2-(7-Fluoro-2,4-dimethyl-1H-indol-3-yl)ethyl]-N′-isoquinolin-1-yl-pyrimidine-4,6-diamine
51. N-Benzothiazol-6-yl-N′-[2-(7-fluoro-2,4-dimethyl-1H-indol-3-yl)ethyl]-pyrimidine-4,6-diamine
52. N-(4-tert-Butylphenyl)-N′-[2-(7-fluoro-2,4-dimethyl-1H-indol-3-yl)ethyl]-pyrimidine-4,6-diamine
53. N-[2-(7-Fluoro-2,4-dimethyl-1H-indol-3-yl)ethyl]-N′-(5-trifluoromethylpyridin-2-yl)pyrimidine-4,6-diamine
54. N-[2-(7-Fluoro-2,4-dimethyl-1H-indol-3-yl)ethyl]-N′-isoquinolin-3-yl-pyrimidine-4,6-diamine
55. (4-{6-[2-(7-Fluoro-2,4-dimethyl-1H-indol-3-yl)ethylamino]pyrimidin-4-ylamino}phenyl)acetonitrile
56. N-[2-(7-Fluoro-2,4-dimethyl-1H-indol-3-yl)ethyl]-N′-(2,4,5,6-tetrahydro-cyclopentapyrazol-3-yl)pyrimidine-4,6-diamine
57. N-(2,3-Dihydrobenzo[1,4]dioxin-6-yl)-N′-[2-(7-fluoro-2,4-dimethyl-1H-indol-3-yl)ethyl]pyrimidine-4,6-diamine
58. N-[2-(7-Fluoro-2,4-dimethyl-1H-indol-3-yl)ethyl]-N′-(4-phenoxyphenyl)-pyrimidine-4,6-diamine
59. 7-{6-[2-(7-Fluoro-2,4-dimethyl-1H-indol-3-yl)ethylamino]pyrimidin-4-ylamino}-4-methylchromen-2-one
60. N-[2-(7-Fluoro-2,4-dimethyl-1H-indol-3-yl)ethyl]-N′-(2-methylbenzothiazol-5-yl)pyrimidine-4,6-diamine
61. [2-(7-Fluoro-2,4-dimethyl-1H-indol-3-yl)ethyl](2-methyl-6-piperidin-1-yl-pyrimidin-4-yl)amine
62. N-Biphenyl-4-yl-N′-[2-(7-fluoro-2,4-dimethyl-1H-indol-3-yl)ethyl]-2-methylpyrimidine-4,6-diamine
63. N-[2-(7-Fluoro-2,4-dimethyl-1H-indol-3-yl)ethyl]-2-methyl-N′-pyridin-2-yl-pyrimidine-4,6-diamine
64. N-[2-(7-Fluoro-2,4-dimethyl-1H-indol-3-yl)ethyl]-2-methyl-N′-pyridin-3-yl-pyrimidine-4,6-diamine
65. N-(3-Chlorophenyl)-N′-[2-(7-fluoro-2,4-dimethyl-1H-indol-3-yl)ethyl]-2-methylpyrimidine-4,6-diamine
66. N-[2-(7-Fluoro-2,4-dimethyl-1H-indol-3-yl)ethyl]-2-methyl-N′-(3-trifluoromethylphenyl)pyrimidine-4,6-diamine
67. N-[2-(7-Fluoro-2,4-dimethyl-1H-indol-3-yl)ethyl]-N′-(4-fluorophenyl)-2-methylpyrimidine-4,6-diamine
68. N-[2-(7-Fluoro-2,4-dimethyl-1H-indol-3-yl)ethyl]-N′-(2-fluorophenyl)-2-methylpyrimidine-4,6-diamine
69. N-[2-(7-Fluoro-2,4-dimethyl-1H-indol-3-yl)ethyl]-2-methyl-N′-(2-trifluoromethylphenyl)pyrimidine-4,6-diamine
70. N-[2-(7-Fluoro-2,4-dimethyl-1H-indol-3-yl)ethyl]-2-methyl-N′-pyridin-4-yl-pyrimidine-4,6-diamine
71. N-[2-(7-Fluoro-2,4-dimethyl-1H-indol-3-yl)ethyl]-2-methyl-N′-phenethyl-pyrimidine-4,6-diamine
72. N-[2-(7-Fluoro-2,4-dimethyl-1H-indol-3-yl)ethyl]-2-methyl-N′-pyridin-2-ylmethylpyrimidine-4,6-diamine
73. N-[2-(7-Fluoro-2,4-dimethyl-1H-indol-3-yl)ethyl]-2-methyl-N′-pyridin-3-ylmethylpyrimidine-4,6-diamine
74. N-[2-(7-Fluoro-2,4-dimethyl-1H-indol-3-yl)ethyl]-2-methyl-N′-pyridin-4-ylmethylpyrimidine-4,6-diamine
75. N-[2-(7-Fluoro-2,4-dimethyl-1H-indol-3-yl)ethyl]-2-methyl-N′-phenyl-pyrimidine-4,6-diamine
76. N-[2-(7-Fluoro-2,4-dimethyl-1H-indol-3-yl)ethyl]-N′-(2-methoxyphenyl)-2-methylpyrimidine-4,6-diamine
77. N-[2-(7-Fluoro-2,4-dimethyl-1H-indol-3-yl)ethyl]-N′-(3-methoxyphenyl)-2-methylpyrimidine-4,6-diamine
78. N-[2-(7-Fluoro-2,4-dimethyl-1H-indol-3-yl)ethyl]-N′-(4-methoxyphenyl)-2-methylpyrimidine-4,6-diamine
79. N-(4-Chlorophenyl)-N′-[2-(7-fluoro-2,4-dimethyl-1H-indol-3-yl)ethyl]-2-methylpyrimidine-4,6-diamine
80. N-Cyclohexyl-N′-[2-(7-fluoro-2,4-dimethyl-1H-indol-3-yl)ethyl]-2-methylpyrimidine-4,6-diamine
81. N-(4-Dimethylaminophenyl)-N′-[2-(7-fluoro-2,4-dimethyl-1H-indol-3-yl)ethyl]-2-methylpyrimidine-4,6-diamine
82. N-[2-(7-Fluoro-2,4-dimethyl-1H-indol-3-yl)ethyl]-2-methyl-N′-pyrazin-2-yl-pyrimidine-4,6-diamine
83. N-Benzyl-N′-[2-(7-fluoro-2,4-dimethyl-1H-indol-3-yl)ethyl]-2-methylpyrimidine-4,6-diamine
84. N-[2-(7-Fluoro-2,4-dimethyl-1H-indol-3-yl)ethyl]-N′-(4-methoxybenzyl)-2-methylpyrimidine-4,6-diamine
85. N-[2-(7-Fluoro-2,4-dimethyl-1H-indol-3-yl)ethyl]-2-methyl-N′-(3-methyl-isothiazol-5-yl)pyrimidine-4,6-diamine
86. [2-(7-Fluoro-2,4-dimethyl-1H-indol-3-yl)ethyl]-[2-methyl-6-(4-pyridin-2-yl-piperazin-1-yl)pyrimidin-4-yl]amine
87. N-Biphenyl-2-ylmethyl-N′-[2-(7-fluoro-2,4-dimethyl-1H-indol-3-yl)ethyl]-2-methylpyrimidine-4,6-diamine
88. [2-(7-Fluoro-2,4-dimethyl-1H-indol-3-yl)ethyl]-[2-methyl-6-(4-pyrimidin-2-yl-piperazin-1-yl)pyrimidin-4-yl]amine
89. [6-(4-Benzylpiperazin-1-yl)-2-methylpyrimidin-4-yl][2-(7-fluoro-2,4-dimethyl-1H-indol-3-yl)ethyl]amine
90. N-[2-(7-Fluoro-2,4-dimethyl-1H-indol-3-yl)ethyl]-2-methyl-N′-(4-[1,2,4]triazol-1-ylphenyl)pyrimidine-4,6-diamine
91. N-(4-{6-[2-(7-Fluoro-2,4-dimethyl-1H-indol-3-yl)ethylamino]-2-methylpyrimidin-4-ylamino}phenyl)acetamide
92. N-(2-Fluorobenzyl)-N′-[2-(7-fluoro-2,4-dimethyl-1H-indol-3-yl)ethyl]-2-methylpyrimidine-4,6-diamine
93. N-Cyclohexylmethyl-N′-[2-(7-fluoro-2,4-dimethyl-1H-indol-3-yl)ethyl]-2-methylpyrimidine-4,6-diamine
94. N-(4-Fluorobenzyl)-N′-[2-(7-fluoro-2,4-dimethyl-1H-indol-3-yl)ethyl]-2-methylpyrimidine-4,6-diamine
95. N-[2-(7-Fluoro-2,4-dimethyl-1H-indol-3-yl)ethyl]-2-methyl-N′-(3-trifluoromethylbenzyl)pyrimidine-4,6-diamine
96. N-[2-(7-Fluoro-2,4-dimethyl-1H-indol-3-yl)ethyl]-2-methyl-N′-(4-methyl-benzyl)pyrimidine-4,6-diamine
97. N-[2-(7-Fluoro-2,4-dimethyl-1H-indol-3-yl)ethyl]-2-methyl-N′-(4-trifluoromethylbenzyl)pyrimidine-4,6-diamine
98. N-[2-(7-Fluoro-2,4-dimethyl-1H-indol-3-yl)ethyl]-2-methyl-N′-(4-trifluoromethylphenyl)pyrimidine-4,6-diamine
99. N-Biphenyl-4-ylmethyl-N′-[2-(7-fluoro-2,4-dimethyl-1H-indol-3-yl)ethyl]-2-methylpyrimidine-4,6-diamine
100. N-Biphenyl-3-ylmethyl-N′-[2-(7-fluoro-2,4-dimethyl-1H-indol-3-yl)ethyl]-2-methylpyrimidine-4,6-diamine
101. 4-{6-[2-(7-Fluoro-2,4-dimethyl-1H-indol-3-yl)ethylamino]-2-methylpyrimidin-4-ylamino}-N-methylbenzamide
102. 4-{6-[2-(7-Fluoro-2,4-dimethyl-1H-indol-3-yl)ethylamino]-2-methylpyrimidin-4-ylamino}-N-thiazol-2-ylbenzenesulfonamide
103. 4-{6-[2-(7-Fluoro-2,4-dimethyl-1H-indol-3-yl)ethylamino]-2-methylpyrimidin-4-ylamino}-N-pyrimidin-2-ylbenzenesulfonamide
104. N-(4,6-Dimethylpyrimidin-2-yl)-4-{6-[2-(7-fluoro-2,4-dimethyl-1H-indol-3-yl)ethylamino]-2-methylpyrimidin-4-ylamino}benzenesulfonamide
105. N-Acetyl-4-{6-[2-(7-fluoro-2,4-dimethyl-1H-indol-3-yl)ethylamino]-2-methylpyrimidin-4-ylamino}benzenesulfonamide
106. N-[2-(7-Fluoro-2,4-dimethyl-1H-indol-3-yl)ethyl]-2-methyl-N′-(6-methylpyridin-2-yl)pyrimidine-4,6-diamine
107. 5-{6-[2-(7-Fluoro-2,4-dimethyl-1H-indol-3-yl)ethylamino]-2-methylpyrimidin-4-ylamino}indan-1-one
108. 6-{6-[2-(7-Fluoro-2,4-dimethyl-1H-indol-3-yl)ethylamino]-2-methylpyrimidin-4-ylamino}-3,4-dihydro-2H-naphthalen-1-one
109. 5-{6-[2-(7-Fluoro-2,4-dimethyl-1H-indol-3-yl)ethylamino]-2-methylpyrimidin-4-ylamino}isoindole-1,3-dione
110. 6-{6-[2-(7-Fluoro-2,4-dimethyl-1H-indol-3-yl)ethylamino]-2-methylpyrimidin-4-ylamino}nicotinamide
111. N-[2-(7-Fluoro-2,4-dimethyl-1H-indol-3-yl)ethyl]-2-methyl-N′-naphthalen-1-ylpyrimidine-4,6-diamine
112. N-Benzo[1,3]dioxol-5-yl-N′-[2-(7-fluoro-2,4-dimethyl-1H-indol-3-yl)ethyl]-2-methylpyrimidine-4,6-diamine
113. N-[2-(7-Fluoro-2,4-dimethyl-1H-indol-3-yl)ethyl]-N′-(1H-indol-5-yl)-2-methylpyrimidine-4,6-diamine
114. N-[2-(7-Fluoro-2,4-dimethyl-1H-indol-3-yl)ethyl]-N′-indan-5-yl-2-methylpyrimidine-4,6-diamine
115. 4-{6-[2-(7-Fluoro-2,4-dimethyl-1H-indol-3-yl)ethylamino]-2-methyl-pyrimidin-4-ylamino}isoindole-1,3-dione
116. 4-{6-[2-(7-Fluoro-2,4-dimethyl-1H-indol-3-yl)ethylamino]-2-methyl-pyrimidin-4-ylamino}benzamide
117. 6-{6-[2-(7-Fluoro-2,4-dimethyl-1H-indol-3-yl)ethylamino]-2-methyl-pyrimidin-4-ylamino}-2,3-dihydrophthalazine-1,4-dione
118. N-[2-(7-Fluoro-2,4-dimethyl-1H-indol-3-yl)ethyl]-2-methyl-N′-(5-methyl-2H-pyrazol-3-yl)pyrimidine-4,6-diamine
119. N-[2-(7-Fluoro-2,4-dimethyl-1H-indol-3-yl)ethyl]-2-methyl-N′-quinolin-3-yl-pyrimidine-4,6-diamine
120. N-[2-(7-Fluoro-2,4-dimethyl-1H-indol-3-yl)ethyl]-2-methyl-N′-quinolin-5-yl-pyrimidine-4,6-diamine
121. N-[2-(7-Fluoro-2,4-dimethyl-1H-indol-3-yl)ethyl]-2-methyl-N′-quinolin-6-yl-pyrimidine-4,6-diamine
122. N-[2-(7-Fluoro-2,4-dimethyl-1H-indol-3-yl)ethyl]-2-methyl-N′-quinolin-8-yl-pyrimidine-4,6-diamine
123. 5-{6-[2-(7-Fluoro-2,4-dimethyl-1H-indol-3-yl)ethylamino]-2-methylpyrimidin-4-ylamino}-2-methylisoindole-1,3-dione
124. N-[2-(7-Fluoro-2,4-dimethyl-1H-indol-3-yl)ethyl]-2-methyl-N′-(1H-pyrazolo[3,4-d]pyrimidin-4-yl)pyrimidine-4,6-diamine
125. N-(2,5-Dimethyl-2H-pyrazol-3-yl)-N′-[2-(7-fluoro-2,4-dimethyl-1H-indol-3-yl)ethyl]-2-methylpyrimidine-4,6-diamine
126. N-[2-(7-Fluoro-2,4-dimethyl-1H-indol-3-yl)ethyl]-2-methyl-N′-(2-trifluoromethyl-1H-benzoimidazol-5-yl)pyrimidine-4,6-diamine
127. N-[2-(7-Fluoro-2,4-dimethyl-1H-indol-3-yl)ethyl]-2-methyl-N′-[3-(1H-tetrazol-5-yl)phenyl]pyrimidine-4,6-diamine
128. N-[2-(7-Fluoro-2,4-dimethyl-1H-indol-3-yl)ethyl]-N′-(6-methoxypyridin-3-yl)-2-methylpyrimidine-4,6-diamine
129. N-[2-(7-Fluoro-2,4-dimethyl-1H-indol-3-yl)ethyl]-2-methyl-N′-[1,3,5]triazin-2-ylpyrimidine-4,6-diamine
130. 3-{6-[2-(7-Fluoro-2,4-dimethyl-1H-indol-3-yl)ethylamino]-2-methyl-pyrimidin-4-ylamino}benzenesulfonamide
131. N-[2-(7-Fluoro-2,4-dimethyl-1H-indol-3-yl)ethyl]-N′-(1H-indazol-5-yl)-2-methylpyrimidine-4,6-diamine
132. N-[2-(7-Fluoro-2,4-dimethyl-1H-indol-3-yl)ethyl]-N′-(1H-indazol-6-yl)-2-methylpyrimidine-4,6-diamine
133. N-[2-(7-Fluoro-2,4-dimethyl-1H-indol-3-yl)ethyl]-N′-isoquinolin-1-yl-2-methylpyrimidine-4,6-diamine
134. N-Benzothiazol-6-yl-N′-[2-(7-fluoro-2,4-dimethyl-1H-indol-3-yl)ethyl]-2-methylpyrimidine-4,6-diamine
135. N-[2-(7-Fluoro-2,4-dimethyl-1H-indol-3-yl)ethyl]-2-methyl-N′-[1,2,4]triazin-3-ylpyrimidine-4,6-diamine
136. N-(4-tert-Butylphenyl)-N′-[2-(7-fluoro-2,4-dimethyl-1H-indol-3-yl)ethyl]-2-methylpyrimidine-4,6-diamine
137. N-[2-(7-Fluoro-2,4-dimethyl-1H-indol-3-yl)ethyl]-2-methyl-N′-(5-trifluoromethylpyridin-2-yl)pyrimidine-4,6-diamine
138. N-[2-(7-Fluoro-2,4-dimethyl-1H-indol-3-yl)ethyl]-N′-isoquinolin-3-yl-2-methylpyrimidine-4,6-diamine
139. N-[2-(7-Fluoro-2,4-dimethyl-1H-indol-3-yl)ethyl]-2-methyl-N′-(2,4,5,6-tetrahydrocyclopentapyrazol-3-yl)pyrimidine-4,6-diamine
140. N-(2,3-Dihydrobenzo[1,4]dioxin-6-yl)-N′-[2-(7-fluoro-2,4-dimethyl-1H-indol-3-yl)ethyl]-2-methylpyrimidine-4,6-diamine
141. N-[2-(7-Fluoro-2,4-dimethyl-1H-indol-3-yl)ethyl]-2-methyl-N′-(4-phenoxy-phenyl)pyrimidine-4,6-diamine
142. 7-{6-[2-(7-Fluoro-2,4-dimethyl-1H-indol-3-yl)ethylamino]-2-methylpyrimidin-4-ylamino}-4-methylchromen-2-one
143. N-[2-(7-Fluoro-2,4-dimethyl-1H-indol-3-yl)ethyl]-2-methyl-N′-(2-methyl-benzothiazol-5-yl)pyrimidine-4,6-diamine

The present invention relates to the use of the compounds of the invention for manufacturing medicaments which comprise at least one of the compounds of formula I.

The present invention likewise relates to medicaments which comprise the compounds of the invention with suitable formulating substances and carriers.

Compared with known prostaglandin E₂ ligands, the novel EP₂ agonists and antagonists are distinguished by greater selectivity and stability.

The present invention relates to medicaments for the treatment and prophylaxis of disorders which include fertility impairments, infectious disorders, cancer, viral infections, cardiovascular disorders, elevated intraocular pressure, glaucoma, skeletal system disorders, angiogenetic disorders, uterine contraction impairments, pain, neuroinflammatory disorders, immunomodulatory infections and nephrological disorders.

Fertility impairments mean the disorders which lead to no ovulation taking place, no fertilization taking place, that the blastocyte development is impaired, that no nidation of a fertilized oocyte occurs and no decidualization takes place, infectious disorders mean disorders caused by unicellular parasites, cancer means solid tumors and leukemia, viral infections mean for example cytomegalievirus infections, hepatitis, hepatitis B and C and HIV disorders, immunomodulatory infections mean for example avian influenza, cardiovascular disorders mean ischemic reperfusion disorder, stenoses, arterioscleroses and restenoses, angiogenetic disorders mean for example endometriosis and fibrosis, elevated intraocular pressure means glaucoma, uterine contraction impairments mean for example painful menstruation, skeletal system disorders mean osteoporosis, neuroinflammatory disorders mean multiple sclerosis, Alzheimer's disease, pain and nephrological disorders mean glomerulonephritis.

The present invention likewise relates to medicaments for the treatment and prophylaxis of the disorders detailed above, which comprise at least one compound of the general formula I, and medicaments with suitable formulating substances and carriers.

For the compounds of the invention to be used as medicaments they are brought into the form of a pharmaceutical product which, besides the active ingredient, comprises inert organic or inorganic pharmaceutical carrier materials which are suitable for enteral or parenteral administration, such as, for example, water, gelatin, gum arabic, lactose, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositiories, emulsions or in liquid form, for example as solutions, suspensions or emulsions.

They comprise where appropriate excipients which are intended to act for example as fillers, binders, disintegrants, lubricants, solvents, solubilizers, masking flavors, colorant, emulsifiers. Examples of types of excipients for the purpose of the invention are saccharides (mono-, di-, tri-, oligo-, and/or polysaccharides), fats, waxes, oils, hydrocarbons, anionic, nonionic, cationic natural, synthetic or semisynthetic surfactants. They additionally comprise where appropriate excipients such as preservatives, stabilizers, wetting agents or emulsifiers; salts to modify the osmotic pressure or buffers. The present invention likewise relates to these pharmaceutical products.

It is expedient to produce aerosol solutions for inhalation.

Suitable for oral use are in particular tablets, coated tablets or capsules with talc and/or hydrocarbon carriers or binders, such as, for example, lactose, corn starch or potato starch. Use can also take place in liquid form, such as, for example, as solution to which, where appropriate, a sweetener is added. Clathrates are likewise also suitable for oral use of such compounds, examples of clathrates which may be mentioned being those with alpha-, beta-, gamma-cyclodextrin or else beta-hydroxypropylcyclodextrin.

Sterile, injectable, aqueous or oily solutions are used for parenteral administration. Particularly suitable are injection solutions or suspensions, especially aqueous solutions of active compounds in polyethoxylated castor oil.

Examples suitable and customary for vaginal administration are pessaries, tampons or intrauterine device.

Appropriately prepared crystal suspensions can be used for intraarticular injection.

It is possible to use for intramuscular injection aqueous and oily injection solutions or suspensions and appropriate depot preparations.

For rectal administration, the novel compounds can be used in the form of suppositories, capsules, solutions (e.g. in the form of enemas) and ointments both for systemic and for local therapy.

The novel compounds can be used in the form of aerosols and inhalations for pulmonary administration.

For local use on the eyes, external auditory canal, middle ear, nasal cavity and paranasal sinuses, the novel compounds can be used as drops, ointments and tinctures in appropriate pharmaceutical preparations.

Formulations possible for topical application are gels, ointments, fatty ointments, creams, pastes, dusting powders, milk and tinctures. The dosage of the compounds of the general formula I should in these preparations be 0.01% -20% in order to achieve an adequate pharmacological effect.

The dosage of the active ingredients may vary depending on the route of administration, age and weight of the patient, nature and severity of the disorder to be treated and similar factors. Treatment can take place by single dosages or by a large number of dosages over a prolonged period. The daily dose is 0.5-1000 mg, preferably 50-200 mg, it being possible to give the dose as a single dose to be administered once or divided into 2 or more daily doses.

Carrier systems which can be used are also surface-active excipients such as salts of bile acids or animal or vegetable phospholipids, but also mixtures thereof, and liposomes or constituents thereof.

The present invention likewise relates to the formulations and dosage forms described above.

Administration of the compounds of the invention can take place by any conventional method, including oral and parenteral, e.g. by subcutaneous or intramuscular injections. The present invention likewise relates to enteral, parenteral, vaginal and oral administrations.

The compounds of the invention of the general formula I bind to the EP₂ receptor and have agonistic or antagonistic effect. It is possible to determine whether an agonistic or an antagonistic effect is present by an agonism test (see Example 1.2.1. of the Biological Examples) or by an antagonism test (see Example 1.2.2. of the Biological Examples).

Antagonists mean molecules which bind to their corresponding receptors and which inhibit the initiation of the signal transduction pathway(s) coupled to the receptor by the naturally occurring ligand(s). The antagonists normally compete with the naturally occurring ligand of the receptor for binding to the receptor.

However, other modifications of the receptor are also possible by molecules which prevent the signal transduction pathways coupled to the receptor being activated by the naturally occurring ligand(s) (e.g. non-competitive, steric modifications of the receptor).

Receptor antagonists typically bind selectively to their particular receptor and not to other receptors. They normally have a higher binding affinity than the natural ligand. Although antagonists which have a higher affinity for the receptor than the natural ligand are preferred, it is likewise possible to employ antagonists having a lower affinity. However, other modifications of the receptor are also possible by molecules which prevent the signal transduction pathways coupled to the receptor being activated by the naturally occurring ligand(s) (e.g. non-competitive, steric modifications of the receptor). The antagonists preferably bind reversibly to their corresponding receptors.

The EP₂ receptor antagonist has a preferred affinity for the EP₂ receptor compared with any other EP receptor. The antagonism is measured in the presence of the natural agonist (PGE₂).

Agonists mean molecules which bind to their corresponding receptors and normally compete with the naturally occurring ligand of the receptor for binding to the receptor, and which stimulate the initiation of the signal transduction pathway coupled to the receptor. Agonists may also assist the binding of the natural ligand.

Receptor agonists typically bind selectively to their particular receptor and not to other receptors. They normally have a higher binding affinity than the natural ligand. Although agonists which have a higher affinity for the receptor than the natural ligand are preferred, it is likewise possible to employ agonists having a lower affinity.

The agonists preferably bind reversibly to their corresponding receptors.

The EP₂ receptor agonist has a preferred affinity for the EP₂ receptor compared with any other EP receptor.

Agonists are tested via the initiation of the signal transduction and/or physiological effect mediated by the corresponding receptor.

The compounds or low molecular weight substances which bind to a receptor are referred to as ligands. Their binding is normally reversible. Binding of a ligand to the corresponding receptor activates or inactivates the signal transduction pathway coupled to the receptor. The ligand mediates its intracellular effect in this manner. Ligands mean agonists and antagonists of a receptor.

The substance of Example 6 shows no inhibition in the cellular agonism test but a good activity (IC₅₀=1.6×10 E-6 M) in the antagonism test.

The present invention likewise relates to the use of the substances of the invention as EP₂ receptor antagonists for the treatment of disorders which are caused by disturbances in the signal transduction chain in which the EP₂ receptor is involved, such as, for example, pain and fertility impairments, and which are likewise suitable for controlling fertility.

The oocyte is surrounded in the preovulatory antral follicle by cumulus cells which form a dense ring of cells around the oocyte. After the lutenizing hormone peak (LH peak), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important constituent of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18):10501-6.) show a distinctly reduced cumulus expansion and severe subfertility, demonstrating the importance of the prostanoid EP₂ receptor for this process.

The substances of the invention have inhibitory effects in cumulus expansion tests.

The present invention relates to the use of the substances of the invention for controlling fertility.

The present invention relates to the use of the substances of the invention for inhibiting cumulus expansion and thus ovulation and fertilization for contraception.

Prostaglandins play an important part in angiogenesis (Sales, Jabbour, 2003, Reproduction 126, 559-567; Kuwano et al., 2004, FASEB J. 18, 300-310;

Kamiyama et al., 2006, Oncogene 25, 7019-7028; Chang et al. 2005, Prostaglandins & other Lipid Mediators 76, 48-58).

Endometriosis is a chronic disorder caused by impairments of blood vessels. About 10% of women regularly suffer from heavy bleeding during menstruation, caused by changes in the blood vessels of the endometrium. In addition, structural differences in the blood vessels have been observed, such as, for example, incomplete formation of the smooth muscle cell layer (Abberton et al., 1999, Hum. Reprod. 14, 1072-1079). Since the blood loss during menstruation is partly controlled by constriction of the blood vessels, it is obvious that the defects in the smooth muscles make a substantial contribution to the bleeding.

The present invention relates to the use of the substances of the general formula I for treating endometriosis.

Prostaglandins play an important part in uterine contraction, and excessively strong contractions are responsible for painful menstruation (Sales, Jabbour, 2003, Reproduction 126, 559-567).

The present invention relates to the use of the substances of the general formula I for the treatment of painful menstruation.

Increasing research results also demonstrate the importance of EP receptors, and especially of the EP₂ receptor, in a large number of types of cancer (e.g. breast cancer, colon carcinoma, lung cancer, prostate cancer, leukemia, skin cancer), suggesting future possibilities of employing modulators (antagonists or agonists) of the EP₂ receptor for the therapy and prevention (prophylactic and/or adjuvant) of cancer (Fulton et al. Cancer Res 2006; 66(20): 9794-7; Castellone et al. Science VOL 310 2005, 1504-1510; Chang et al. Cancer Res 2005; 65(11): 4496-9); Hull et al. Mol Cancer Ther 2004; 3(8):1031-9; Richards et al. J Clin Endocrinol Metab 88: 2810-2816, 2003; Sinha et al. 2007, Cancer Res; 67(9):4507-13; Wang et al. 2004, Seminars in Oncology, Vol 31, No 1, Suppl 3: pp 64-73), Jain et al. Cancer Res 2006; 66(13): 6638-48)).

The present invention relates to the use of the substances of the general formula I for the treatment and prevention of cancers.

Prostaglandins also play an important part in processes counteracting osteoporosis. The present invention therefore relates to the use of the substances of the invention for the treatment of osteoporosis.

Reinold et al. (J. Clin. Invest. 115, 673-679 (2005)) describes PGE₂ receptors of the EP₂ subtype as the key signaling elements in inflammatory hyperalgesia. Mice no longer having this receptor (EP₂^{-/-}) do not experience spinal inflammatory pain. There is evidence that an inflammatory, increased pain sensitivity can be treated by targeted modulation of EP₂ receptors.

The present invention relates to the use of the substances of the invention for the treatment of inflammatory hyperalgesia.

Prostaglandins are important mediators of inflammatory processes. Recent research results show the involvement of the EP₂ receptor in inflammatory bowel diseases (e.g. Crohn's disease): Sheibanie et al. The Journal of Immunology, 2007, 178: 8138-8147.

The present invention relates to the use of the substances of the invention for the treatment of inflammatory disorders, for example inflammatory bowel diseases, such as Crohn's disease.

The invention additionally relates to a process for preparing the compounds of the invention of the general formula I, which comprises reacting a compound of the general formula IV

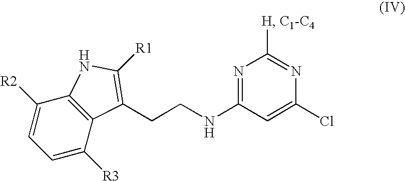

in which R¹, R² and R³ have the meanings indicated above, with an amine of the general formula V

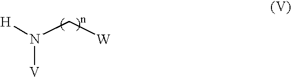

in which V and W have the meanings indicated above by methods known to the skilled worker.

The reaction of the chloropyrimidine of the general formula IV with an amine of the general formula V can take place in an inert solvent or solvent mixture such as, for example, N,N-dimethylformamide, N,N-dimethylacetamide, toluene, n-butanol, tetrahydrofuran, where appropriate with the addition of an auxiliary base such as, for example, N,N-dimethylaminopyridine, diisopropylethylamine, triethylamine, at temperatures between +20° C. and +165° C., preferably at 60° C. to 120° C.

A further possibility consists of carrying out the reaction of the chloropyrimidine of the general formula IV with an amine of the general formula V in an inert solvent or solvent mixture such as, for example, N-methylpyrrolidinone, toluene with palladium catalysis (with, for example, Pd(OAc)₂, Pd(PPh₃)₄, Pd₂(dba)₃, PdCl₂(dppf)) and addition of a base such as, for example, sodium tert-butoxide and of a suitable ligand such as, for example, 2,2′-bis(diphenylphosphino)-1,1′-binaphthyl at temperatures between +40° C. and +150° C.

In the case where n=0, W=aryl or heteroaryl in the meanings indicated above, and V=H, a further possibility consists of carrying out the reaction of the chloropyrimidine of the general formula IV with the appropriate amine in an inert solvent or solvent mixture such as, for example, n-butanol, acetonitrile with addition of an acid such as, for example, hydrochloric acid, trifluoroacetic acid, at temperatures between +40° C. and +120° C.

The salts are prepared in a conventional way by mixing a solution of the compound of the formula I with the equivalent amount or an excess of a base or acid, which is in solution where appropriate, and separating off the precipitate or working up the solution in a conventional way.

The invention thus also relates to medicaments based on compounds of the general formula I and usual excipients or carriers.

Where the preparation of the starting compounds is not described, they are known or can be prepared in analogy to known compounds or processes described herein. It is likewise possible to carry out all the reactions described herein in parallel reactors or using combinatorial techniques.

The compounds of the invention of the general formula I can be prepared as described in the examples.

Starting from 4,6-dichloropyrimidines of the general formula II, the compounds of the general formula IV can be prepared by reacting with tryptamines of the general formula III by methods known to the skilled worker (scheme 1).

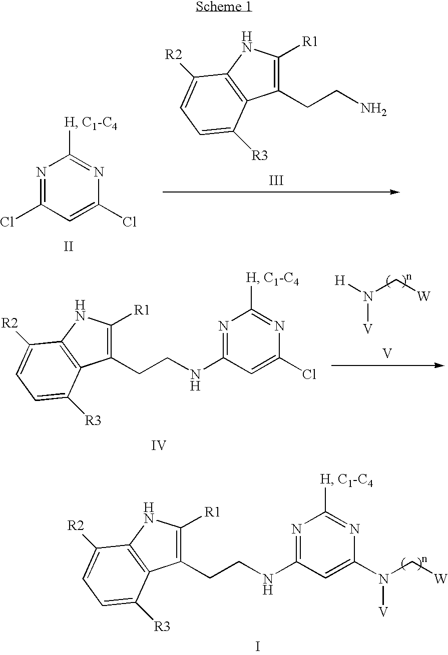

The tryptamines of the general formula III are either known or can be prepared for example by reacting in a manner known per se the known hydrazines VI, where appropriate prepared from the corresponding known anilines by nitrosation followed by a reduction,

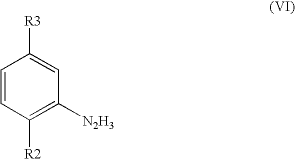

in which R² and R³ have the meaning indicated above,
a) with a ketone of the general formula VII in which R¹ has the meaning indicated above, in a Fischer indole cyclization

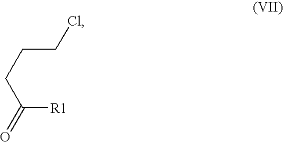

or
b) with an enol ether of the general formula VIII in which R¹ has the meaning indicated above, in a Fischer indole cyclization (Org. Lett. 2004, 79ff),

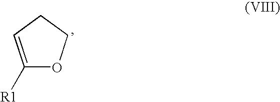

and converting the subsequently obtained alcohol by methods known to the skilled worker by conversion into a leaving group such as tosylate, mesylate, trifluoromesylate, chloride, bromide or iodide and subsequent reaction with, for example, sodium azide followed by a hydrolysis with PPh₃/H₂O in tetrahydrofuran into the amino function.

The compounds of the invention of the general formula I can be prepared by reacting compounds of the general formula IV with amines of the general formula V by processes known to the skilled worker (scheme 1). The further compounds of the general formula I can be obtained by an analogous procedure using homologous reagents to the reagents described in the examples.

The substituents on the radical W of the compounds of the general formula I obtained in this way can be converted by methods known to the skilled worker further into diverse functional groups and thus further compounds of the general formula I.

For example, a bromide or chloride can be replaced by means of palladium(0)-catalyzed reactions by an aryl or heteroaryl ring, a substituted alkene or alkyne, amine or a cyano group.

A carboxy function, cyano group or an amine can be converted into esters and amides of the general formula I for example by methods known to the skilled worker.

It is likewise possible for example to convert ester functions or a cyano group in compounds of the general formula I after reduction to the aldehyde by methods known to the skilled worker into further olefins or secondary alcohols substituted by alkyl or aryl radicals. It is likewise possible for a cyano group in compounds of the general formula I to be converted by methods known to the skilled worker into ketones which are substituted by alkyl or aryl radicals and which can then be reduced to the corresponding secondary alcohols or else can be converted by methods known to the skilled worker into tertiary alcohols substituted by alkyl or aryl radicals.

Abbreviations frequently used:

M molar

### DMF N,N-dimethylformamide

eq equivalents
DIPEA diisopropylethylamine
MTBE tert-butyl methyl ether
NaCl sodium chloride
sat. saturated

### NMP N-methylpyrrolidinone

dba dibenzylideneacetone
NaOtBu sodium tert-butoxide
BINAP 2,2′-bis(diphenylphosphino)-1,1′-binaphthyl

The following examples serve to explain the invention in more detail:

General Procedure for Synthesizing the Compounds of the General Formula IV by Reacting Pyrimidines II with Tryptamines III

The appropriate tryptamine III is introduced 0.3 M into DMF, 1.2 eq of dichloropyrimidine II and 4 eq of DIPEA are added, and the mixture is stirred at room temperature until conversion of the tryptamine III is complete. The reaction mixture is poured into water, extracted several times with MTBE and washed with sat. NaCl solution, and the solvent is removed in vacuo. Purification takes place by column chromatography on silica gel with a hexane/ethyl acetate gradient, and the compounds of the general formula IV are obtained.

(6-Chloropyrimidin-4-yl)-[2-(7-fluoro-2,4-dimethyl-1H-indol-3-yl)ethyl]amine IVa

NMR (300 MHz, DMSO-d6): δ=2.23 (3H), 2.54 (3H), 2.92 (2H), 3.41 (2H), 8.25 (1H)

(6-Chloro-2-methylpyrimidin-4-yl)-[2-(7-fluoro-2,4-dimethyl-1H-indol-3-yl)ethyl]-amine IVb

NMR (300 MHz, DMSO-d6): δ=2.27 (6H), 2.59 (3H), 2.91 (2H), 3.37 (2H), 8.25 (1H)

General Procedure for Synthesizing Compounds of the General Formula I by Hartwig-Buchwald Coupling of the Compounds of the type IV with Amines V

The appropriate compound IV is introduced 0.2 M into NMP, 1.5 eq of amine V (0.4 M in NMP), 0.2 eq of palladium catalyst Pd₂(dba)₃ (0.014 M in NMP), 2.5 eq of NaOtBu (1 M in NMP) and 0.6 eq of rac-BINAP (0.1 M in NMP) are added, and the reaction mixture is heated at 150° C. for 1 hour. After cooling, the reaction mixture is concentrated in vacuo and purified by means of preparative HPLC (analytical 4-channel MUX system with CTC Pal injector, Waters 1525 pumps, Waters 2488 UV detector and Waters ZQ 2000 single quad MS detector, column X-Bridge RP C18 4.6x50 3.5 μm; detection wavelength 214 nm; flow rate 2 ml/min; eluents A: 0.1% TFA in H₂O, B 0.1% TFA in ACN; gradient in each case based on B: 1% to 99% (5′) to 99% (1′) to 1% (0.25°) to 1% (1.75°), MS: (M+H)⁺).

The following compounds were synthesized by way of example according to this general reaction procedure: 1-143.

| | | HPLC | | |
|---|---|---|---|---|
| | | Retention | MW | MW |
| Example | Structure | time | (calc.) | (found) |
| 1 | 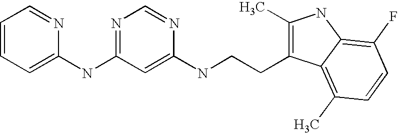 | 3.34 | 376.4369 | 377 |
| 2 | 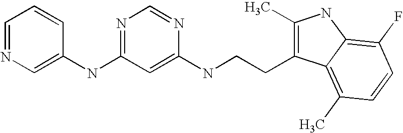 | 2.56 | 376.4369 | 377 |
| 3 | 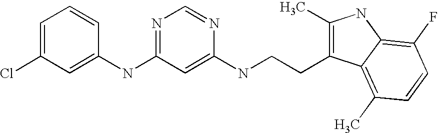 | 3.54 | 409.8939 | 411 |
| 4 | 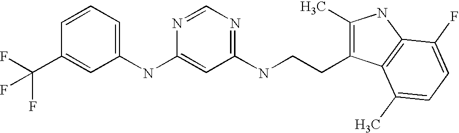 | 3.77 | 443.4459 | 444 |
| 5 | 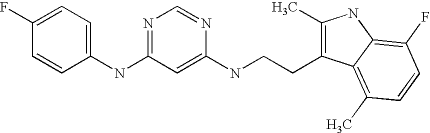 | 3.49 | 393.4389 | 394 |
| 6 | 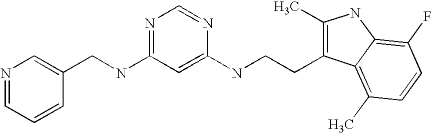 | 2.52 | 390.4637 | 391 |
| 7 | 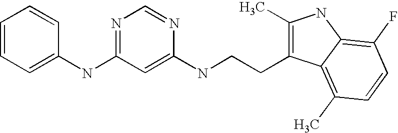 | 3.46 | 375.4488 | 376 |
| 8 | 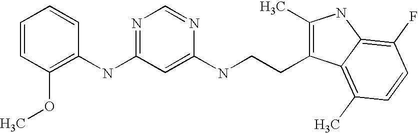 | 3.41 | 405.4746 | 406 |
| 9 | 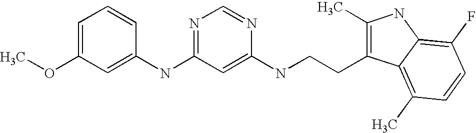 | 3.39 | 405.4746 | 406 |
| 10 | 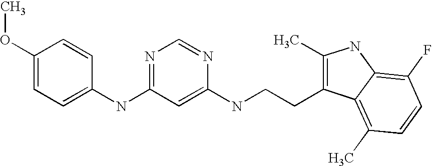 | 3.54 | 405.4746 | 406 |
| 11 | 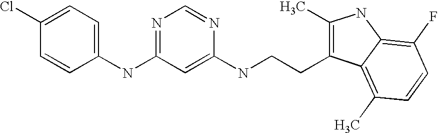 | 3.67 | 409.8939 | 411 |
| 12 | 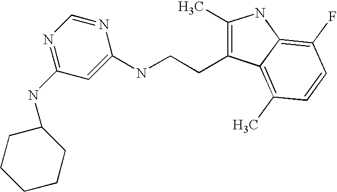 | 3.59 | 381.4962 | 382 |
| 13 | 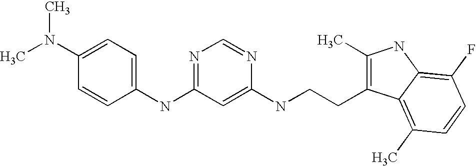 | 2.81 | 418.5173 | 420 |
| 14 | 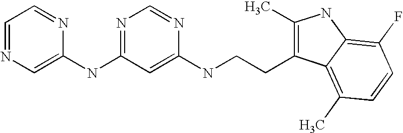 | 3.17 | 377.425 | 378 |
| 15 | 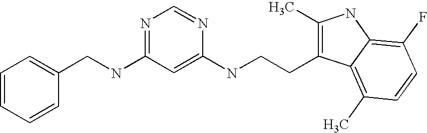 | 3.52 | 389.4756 | 390 |
| 16 | 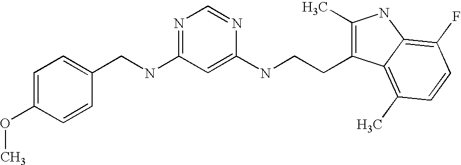 | 3.46 | 419.5014 | 421 |
| 17 | 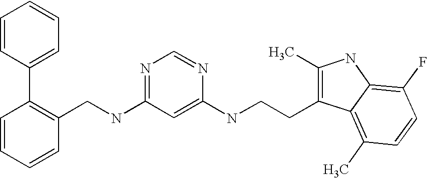 | 3.95 | 465.5732 | 467 |
| 18 | 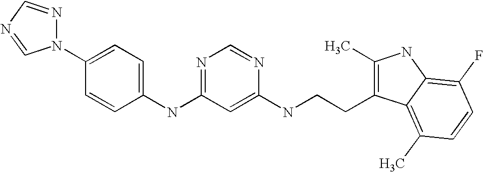 | 2.96 | 442.4997 | 443 |
| 19 | 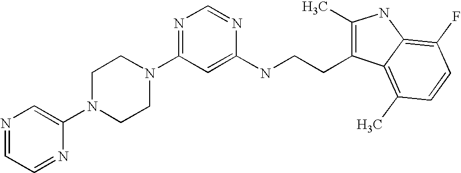 | 3.22 | 446.5313 | 448 |
| 20 | 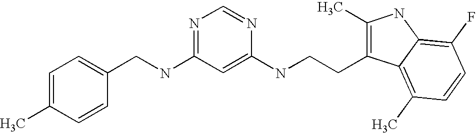 | 3.63 | 403.5024 | 405 |
| 21 | 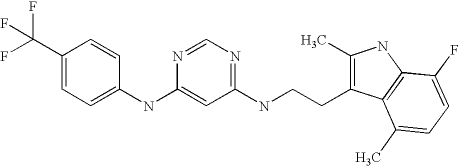 | 3.84 | 443.4459 | 444 |
| 22 | 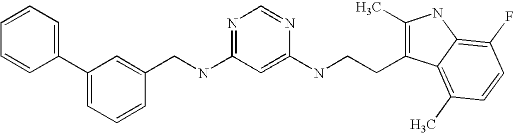 | 3.89 | 465.5732 | 467 |
| 23 | 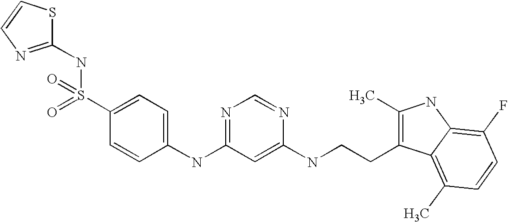 | 2.94 | 537.6416 | 539 |
| 24 | 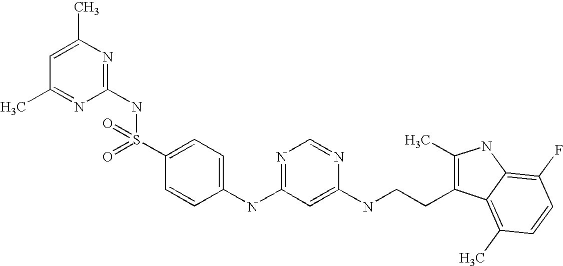 | 3.02 | 560.6551 | 562 |
| 25 | 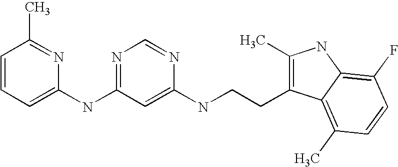 | 3.42 | 390.4637 | 391 |
| 26 | 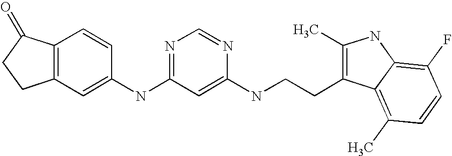 | 3.24 | 429.4966 | 430 |
| 27 | 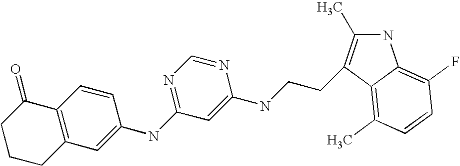 | 3.36 | 443.5234 | 445 |
| 28 | 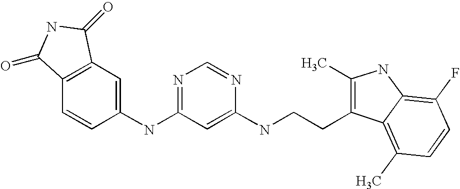 | 2.92 | 444.4679 | 445 |
| 29 | 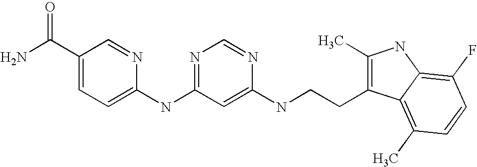 | 2.99 | 419.4618 | 420 |
| 30 | 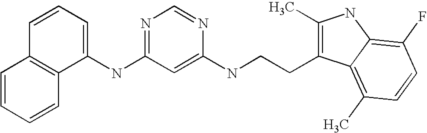 | 3.74 | 425.5086 | 427 |
| 31 | 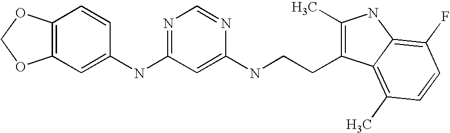 | 3.56 | 419.4578 | 420 |
| 32 | 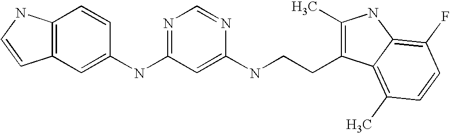 | 3.33 | 414.4857 | 415 |
| 33 | 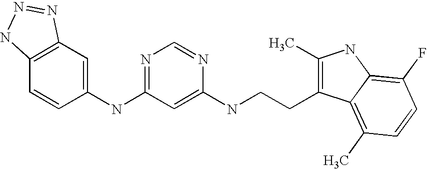 | 2.81 | 416.4619 | 417 |
| 34 | 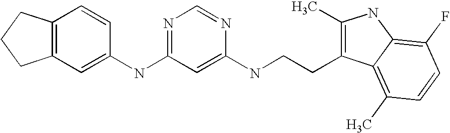 | 3.89 | 415.5134 | 417 |
| 35 | 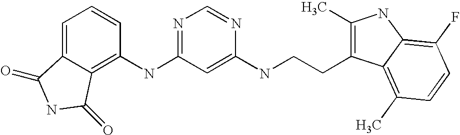 | 3.11 | 444.4679 | 445 |
| 36 | 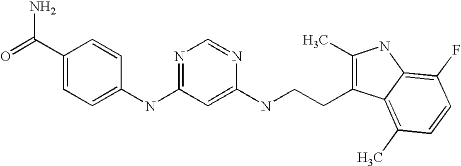 | 3.23 | 418.4737 | 419 |
| 37 | 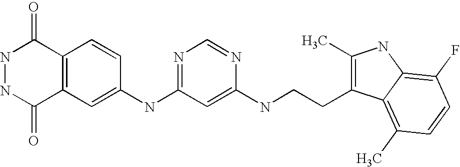 | 3.31 | 459.4828 | 460 |
| 38 | 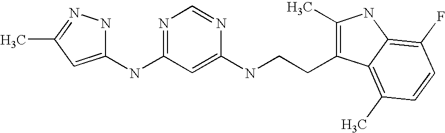 | 3.12 | 379.4408 | 380 |
| 39 | 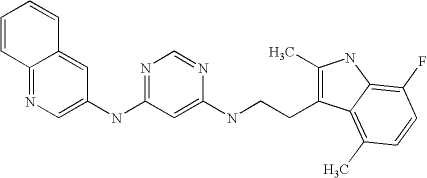 | 3.03 | 426.4967 | 427 |
| 40 | 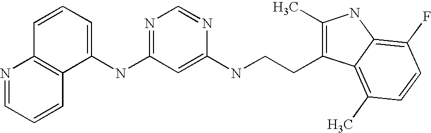 | 2.64 | 426.4967 | 427 |
| 41 | 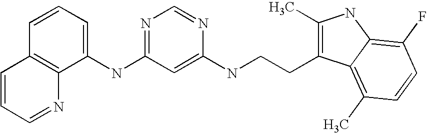 | 3.42 | 426.4967 | 427 |
| 42 | 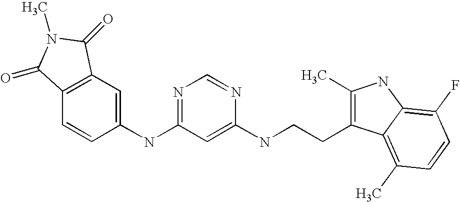 | 3.22 | 458.4947 | 459 |
| 43 | 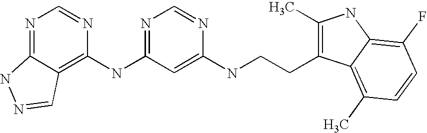 | 3.01 | 417.45 | 418 |
| 44 | 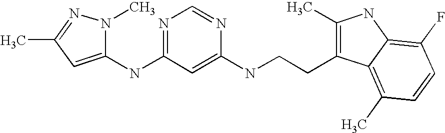 | 2.9 | 393.4676 | 394 |
| 45 | 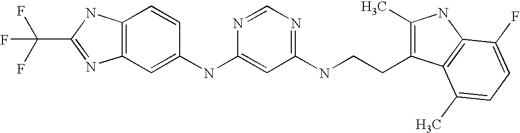 | 3.3 | 483.4709 | 484 |
| 46 | 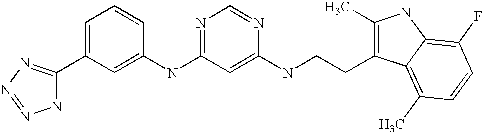 | 3.09 | 443.4878 | 444 |
| 47 | 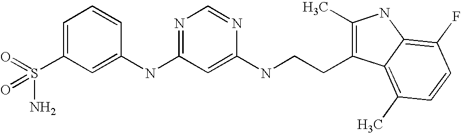 | 2.99 | 454.5277 | 456 |
| 48 | 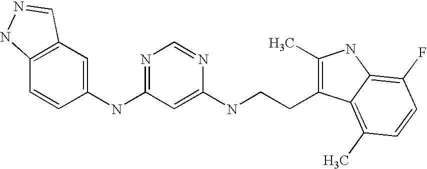 | 2.96 | 415.4738 | 416 |
| 49 | 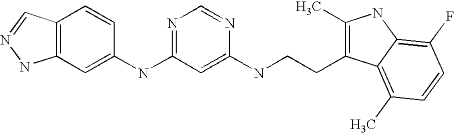 | 3.17 | 415.4738 | 416 |
| 50 | 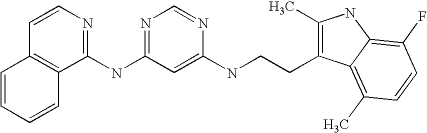 | 3.81 | 426.4967 | 427 |
| 51 | 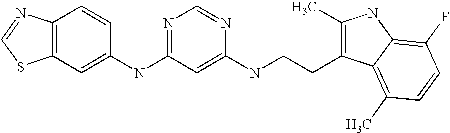 | 3.11 | 432.5249 | 434 |
| 52 | 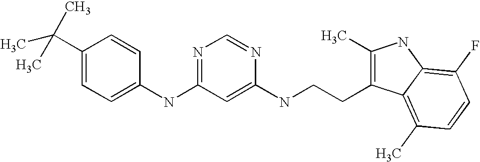 | 3.94 | 431.556 | 433 |
| 53 | 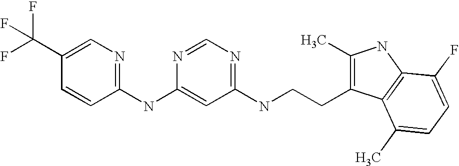 | 3.79 | 444.434 | 445 |
| 54 | 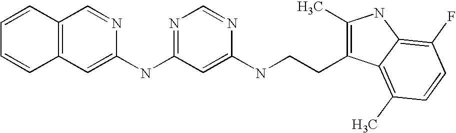 | 3.77 | 426.4967 | 427 |
| 55 | 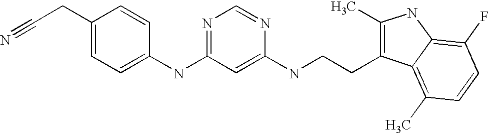 | 3.26 | 414.4857 | 415 |
| 56 | 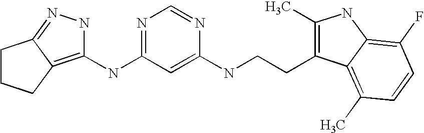 | 3.61 | 405.4786 | 406 |
| 57 | 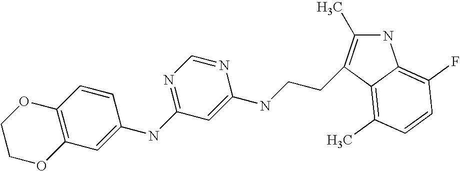 | 3.43 | 433.4846 | 434 |
| 58 | 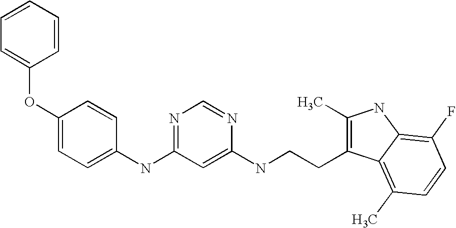 | 3.94 | 467.5454 | 469 |
| 59 | 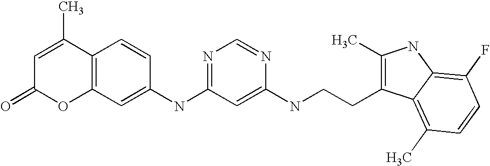 | 3.21 | 457.5066 | 459 |
| 60 | 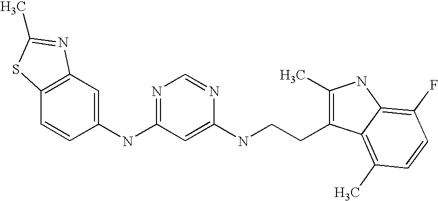 | 3.26 | 446.5517 | 448 |
| 61 | 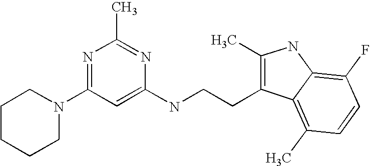 | 3.85 | 381.4962 | 382 |
| 62 | 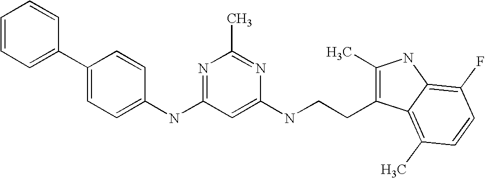 | 4.2 | 465.5732 | 467 |
| 63 | 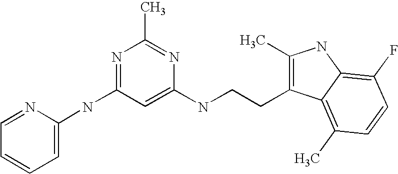 | 3.48 | 390.4637 | 391 |
| 64 | 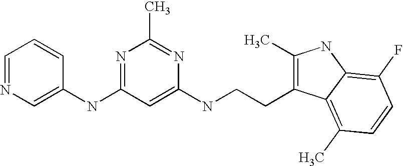 | 2.64 | 390.4637 | 391 |
| 65 | 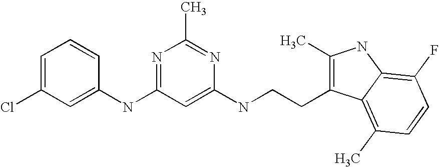 | 3.86 | 423.9207 | 425 |
| 66 | 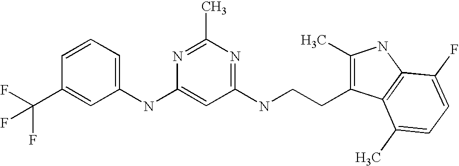 | 3.99 | 457.4727 | 458 |
| 67 | 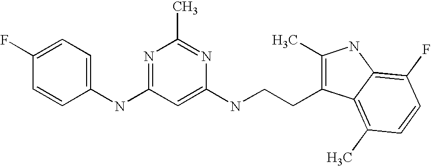 | 3.53 | 407.4657 | 408 |
| 68 | 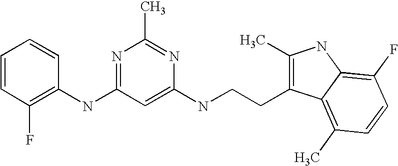 | 3.52 | 407.4657 | 408 |
| 69 | 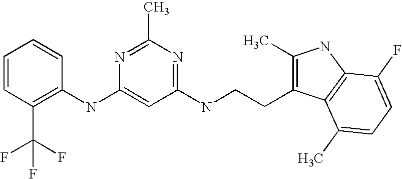 | 3.61 | 457.4727 | 458 |
| 70 | 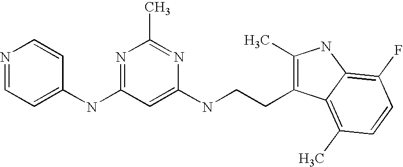 | 2.96 | 390.4637 | 391 |
| 71 | 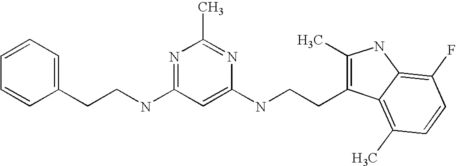 | 3.69 | 417.5292 | 419 |
| 72 | 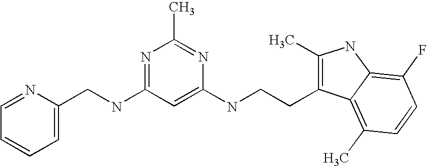 | 2.82 | 404.4905 | 405 |
| 73 | 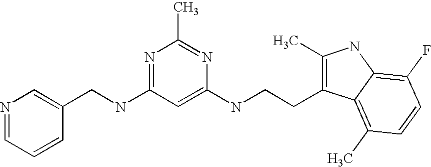 | 2.57 | 404.4905 | 405 |
| 74 | 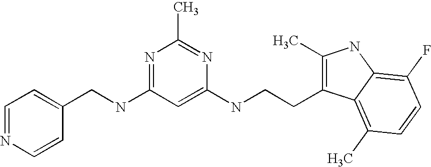 | 2.77 | 404.4905 | 405 |
| 75 | 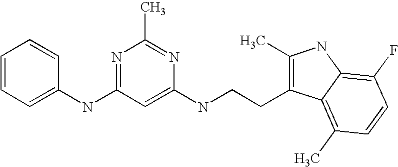 | 3.53 | 389.4756 | 390 |
| 76 | 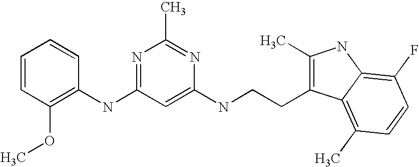 | 3.56 | 419.5014 | 421 |
| 77 | 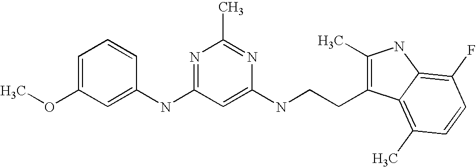 | 3.62 | 419.5014 | 421 |
| 78 | 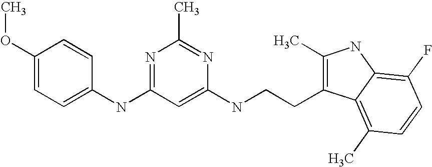 | 3.79 | 419.5014 | 421 |
| 79 | 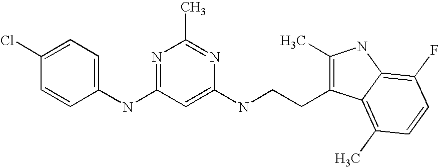 | 3.73 | | 425 |
| 80 | 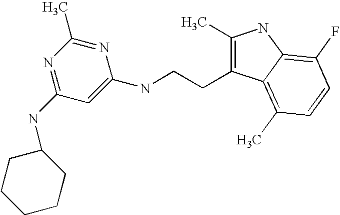 | 3.77 | 395.523 | 397 |
| 81 | 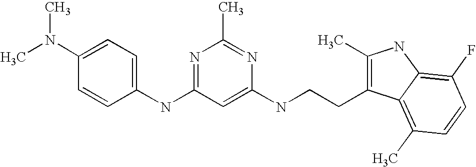 | 3.01 | 432.5441 | 434 |
| 82 | 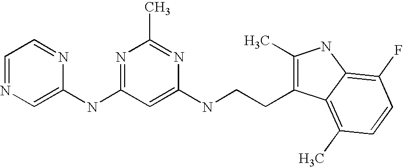 | 3.16 | 391.4518 | 392 |
| 83 | 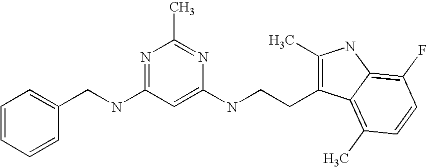 | 3.61 | 403.5024 | 405 |
| 84 | 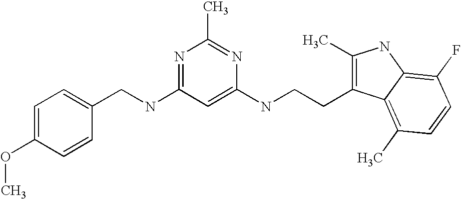 | 3.54 | 433.5282 | 435 |
| 85 | 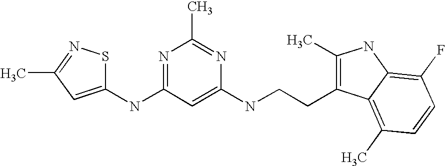 | 3.36 | 410.5187 | 412 |
| 86 | 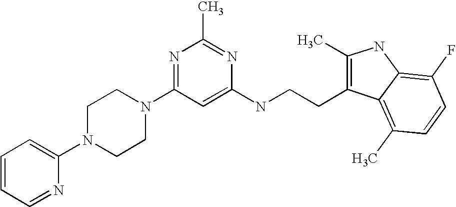 | 2.64 | 459.57 | 461 |
| 87 | 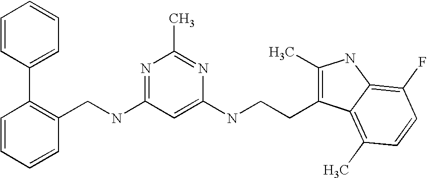 | 3.99 | 479.6 | 481 |
| 88 | 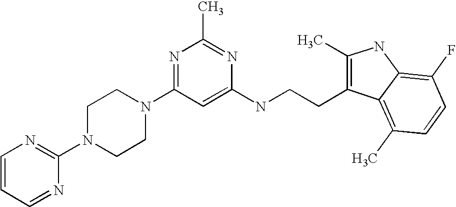 | 3.24 | 460.5581 | 462 |
| 89 | 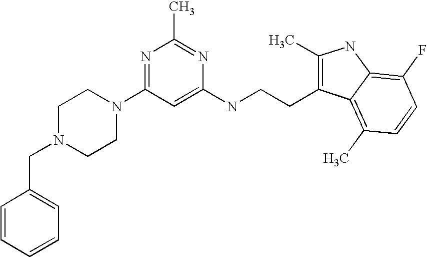 | 3.15 | 472.6087 | 474 |
| 90 | 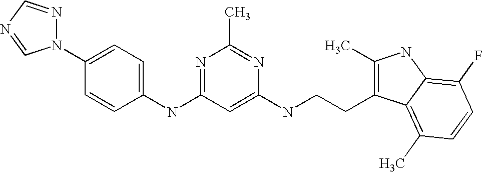 | 3.12 | 456.5265 | 458 |
| 91 | 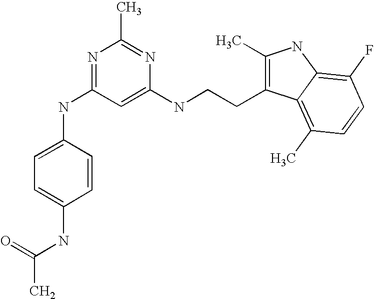 | 3.06 | 446.5273 | 448 |
| 92 | 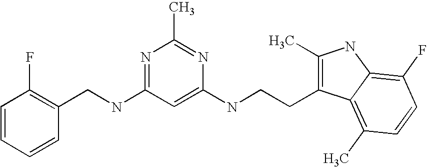 | 3.61 | 421.4925 | 422 |
| 93 | 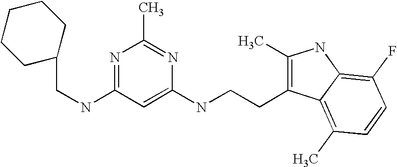 | 4.26 | 409.5498 | 411 |
| 94 | 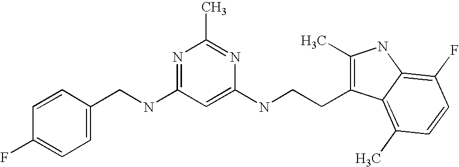 | 3.76 | 421.4925 | 422 |
| 95 | 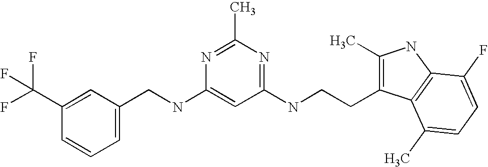 | 3.84 | 471.4995 | 472 |
| 96 | 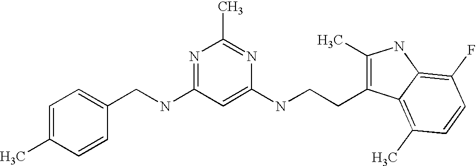 | 3.73 | 417.5292 | 419 |
| 97 | 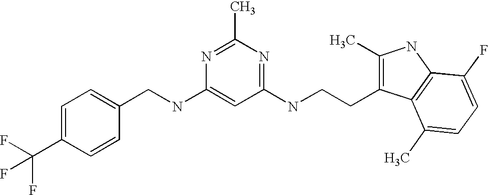 | 3.97 | 471.4995 | 472 |
| 98 | 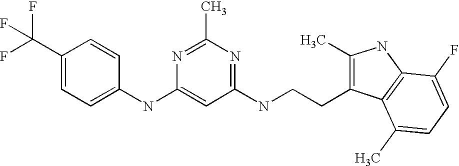 | 3.83 | 457.4727 | 458 |
| 99 | 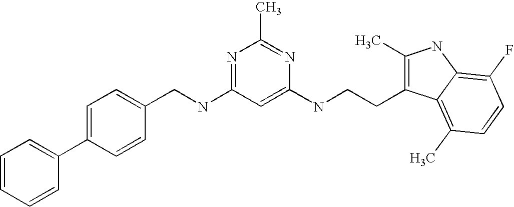 | 4.04 | 479.6 | 481 |
| 100 | 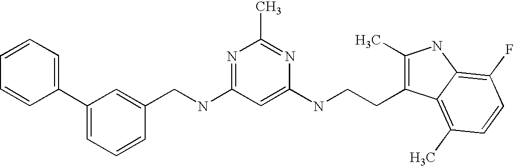 | 3.99 | 479.6 | 481 |
| 101 | 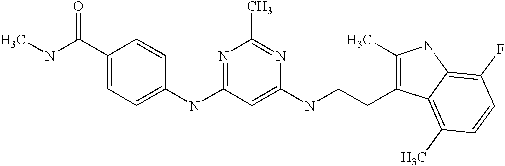 | 3.09 | 446.5273 | 448 |
| 102 | 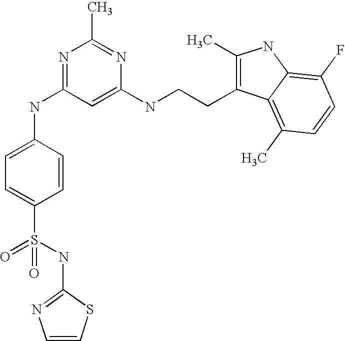 | 3.04 | 551.6684 | 553 |
| 103 | 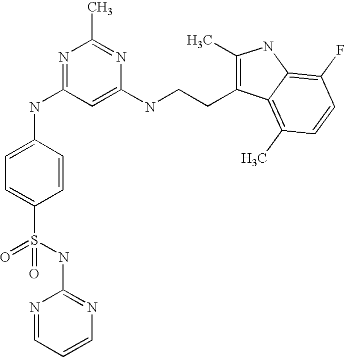 | 3.11 | 546.6283 | 548 |
| 104 | 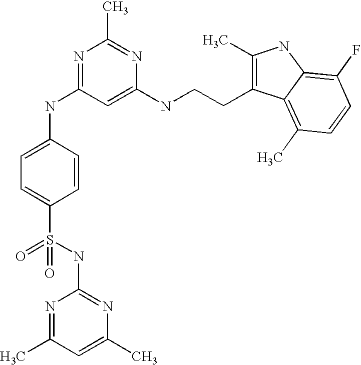 | 3.17 | 574.6819 | 576 |
| 105 | 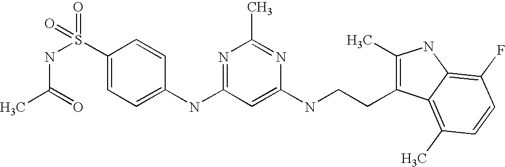 | 3.22 | 510.5913 | 512 |
| 106 | 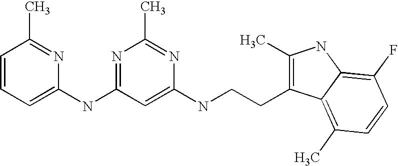 | 3.66 | 404.4905 | 405 |
| 107 | 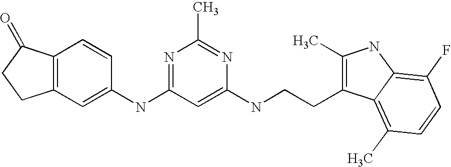 | 3.24 | 443.5234 | 445 |
| 108 | 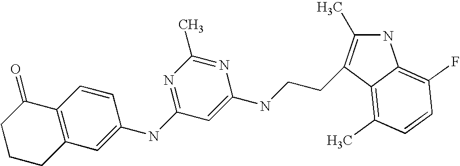 | 3.52 | 457.5502 | 459 |
| 109 | 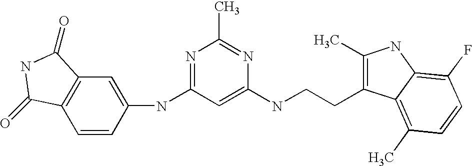 | 3.14 | 458.4947 | 459 |
| 110 | 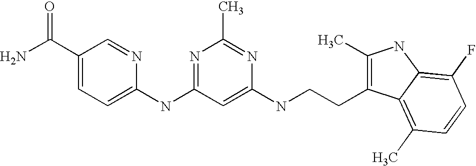 | 3.07 | 433.4886 | 434 |
| 111 | 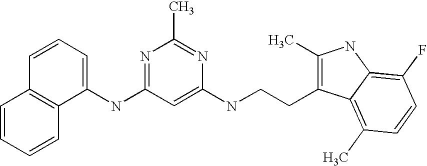 | 3.73 | 439.5354 | 441 |
| 112 | 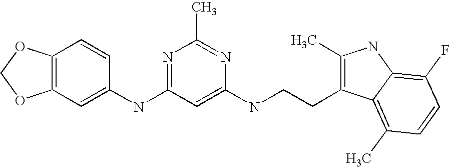 | 3.57 | 433.4846 | 434 |
| 113 | 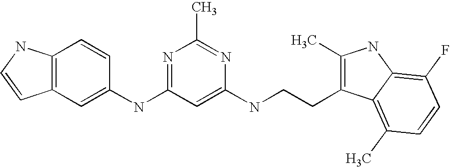 | 3.44 | 428.5125 | 430 |
| 114 | 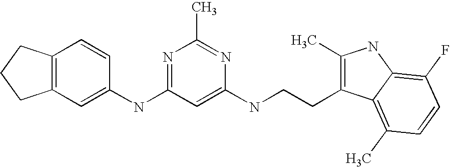 | 3.86 | 429.5402 | 431 |
| 115 | 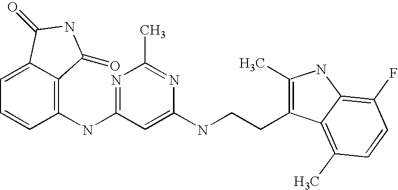 | 3.24 | 458.4947 | 459 |
| 116 | 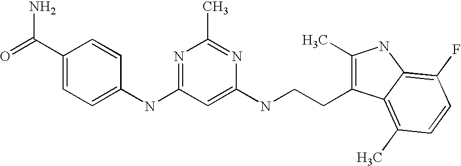 | 3.38 | 432.5005 | 434 |
| 117 | 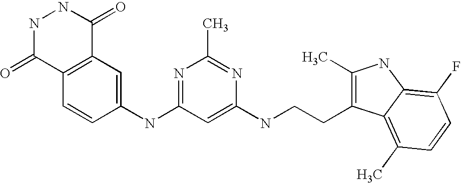 | 3.49 | 473.5096 | 475 |
| 118 | 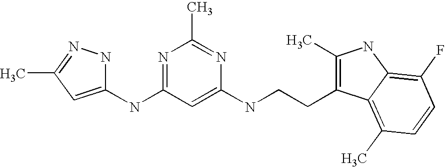 | 3.42 | 393.4676 | 394 |
| 119 | 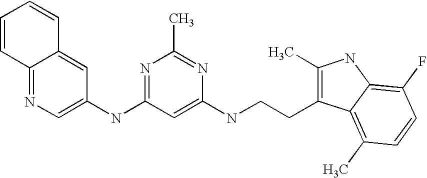 | 3.34 | 440.5235 | 442 |
| 120 | 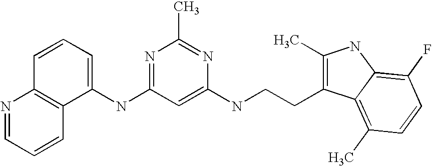 | 2.71 | 440.5235 | 442 |
| 121 | 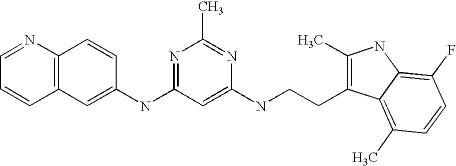 | 2.69 | 440.5235 | 442 |
| 122 | 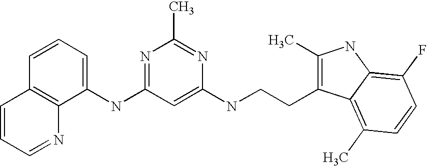 | 3.61 | 440.5235 | 442 |
| 123 | 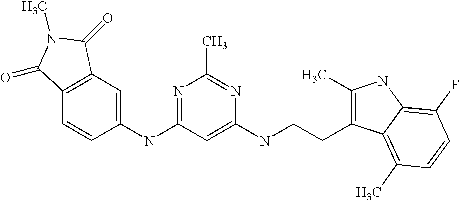 | 3.4 | 472.5215 | 474 |
| 124 | 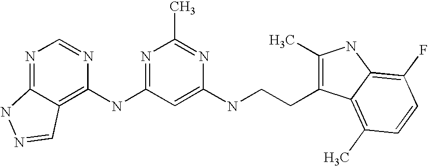 | 3.11 | 431.4768 | 432 |
| 125 | 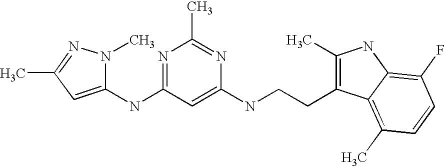 | 3.04 | 407.4944 | 408 |
| 126 | 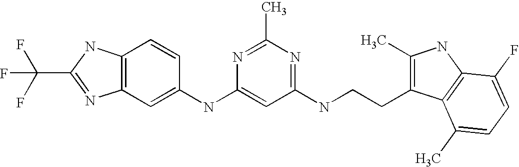 | 3.29 | 497.4977 | 498 |
| 127 | 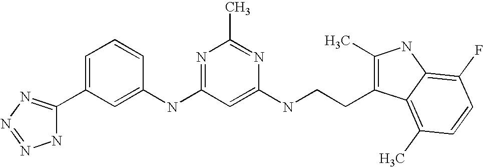 | 3.29 | 457.5146 | 459 |
| 128 | 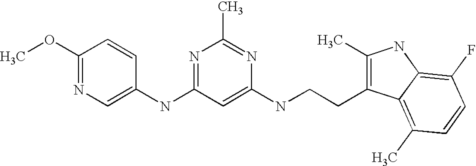 | 3.28 | 420.4895 | 421 |
| 129 | 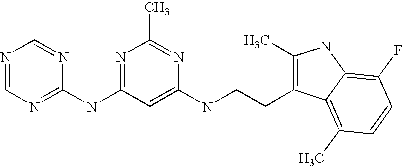 | 2.96 | 392.4399 | 393 |
| 130 | 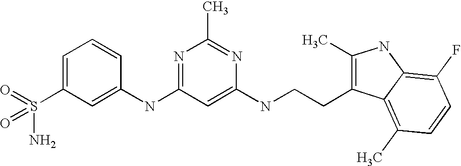 | 3.09 | 468.5545 | 470 |
| 131 | 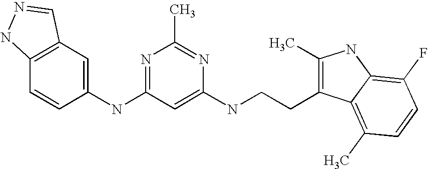 | 3.26 | 429.5006 | 431 |
| 132 | 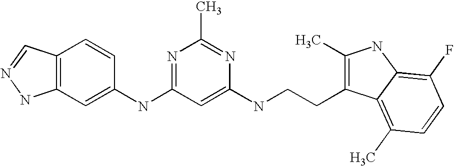 | 3.16 | 429.5006 | 431 |
| 133 | 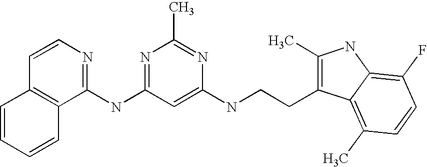 | 3.76 | 440.5235 | 442 |
| 134 | 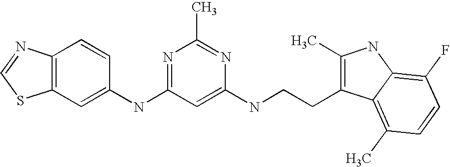 | 3.34 | 446.5517 | 448 |
| 135 | 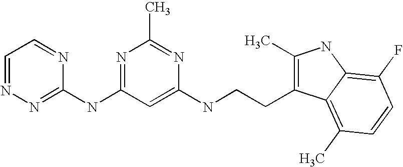 | 3.11 | 392.4399 | 393 |
| 136 | 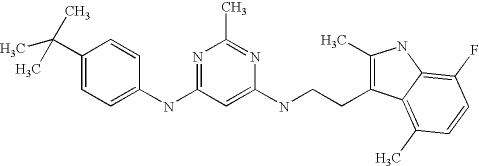 | 4.08 | 445.5828 | 447 |
| 137 | 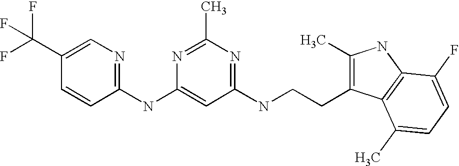 | 3.86 | 458.4608 | 459 |
| 138 | 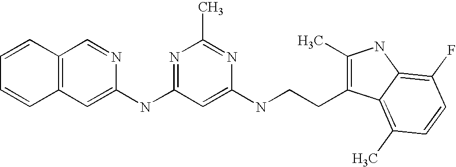 | 3.97 | 440.5235 | 442 |
| 139 | 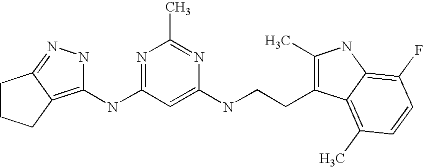 | 3.49 | 419.5054 | 421 |
| 140 | 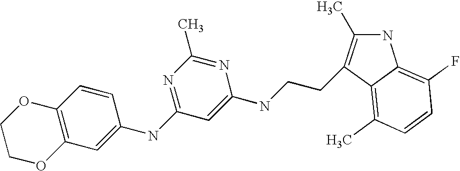 | 3.49 | 447.5114 | 449 |
| 141 | 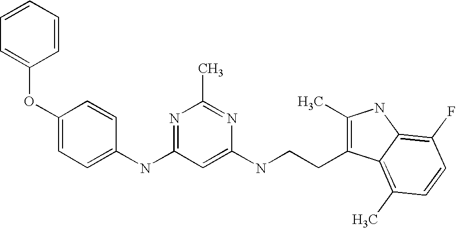 | 3.98 | 481.5722 | 483 |
| 142 | 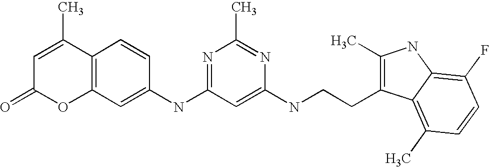 | 3.37 | 471.5334 | 473 |
| 143 | 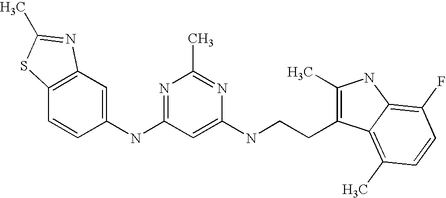 | 3.39 | 460.5785 | 462 |
| 144 | 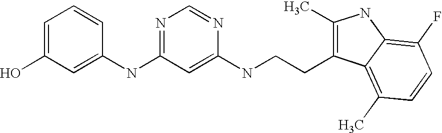 | 0.89 | 391.443 | 392 |
| 145 | 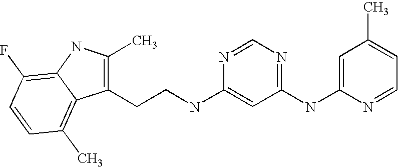 | 1.02 | 390.459 | 391 |
| 146 | 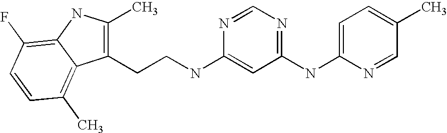 | 1.05 | 390.459 | 391 |
| 147 | 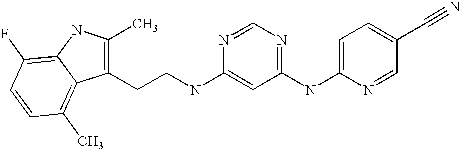 | 0.97 | 401.442 | 402 |
| 148 | 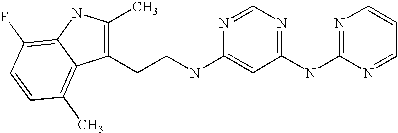 | 0.94 | 377.42 | 378 |
| 149 | 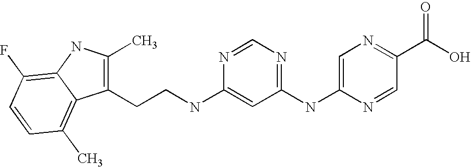 | 0.88 | 421.43 | 422 |
| 150 | 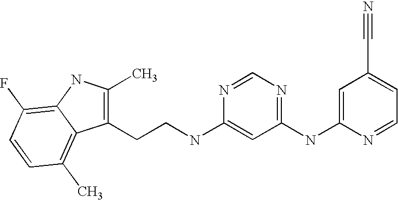 | 0.98 | 401.442 | 402 |
| 151 | 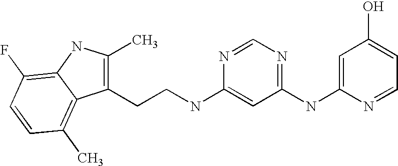 | 0.93 | 392.431 | 393 |
| 152 | 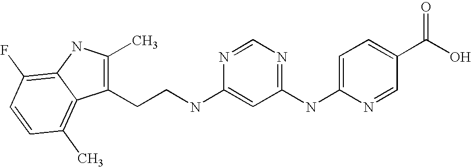 | 0.9 | 420.442 | 421 |
| 153 | 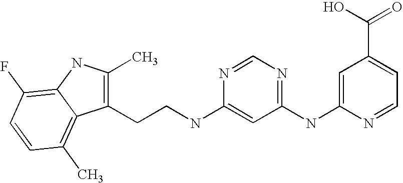 | 0.93 | 420.442 | 421 |
| 154 | 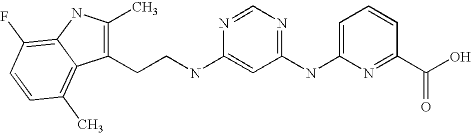 | 0.91 | 420.442 | 421 |
| 155 | 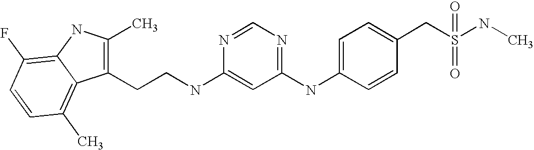 | 0.93 | 482.576 | 483 |
| 156 | 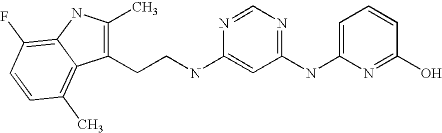 | 0.98 | 392.431 | 393 |
| 157 | 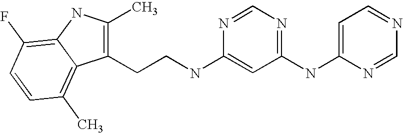 | 0.89 | 377.42 | 378 |
| 158 | 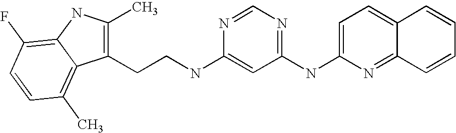 | 1.1 | 426.491 | 427 |
| 159 | 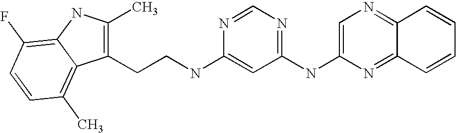 | 1.08 | 427.479 | 428 |
| 160 | 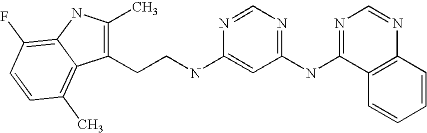 | 0.98 | 427.479 | 428 |
| 161 | 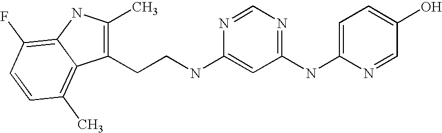 | 0.8 | 392.431 | 393 |
| 162 | 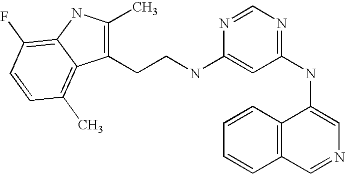 | 0.82 | 426.491 | 427 |
| 163 | 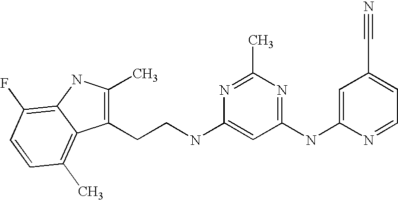 | 0.85 | 415.468 | 416 |
| 164 | 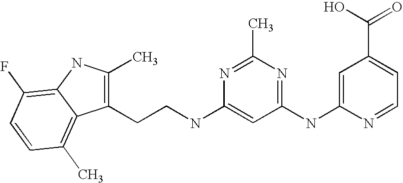 | 0.82 | 434.468 | 435 |
| 165 | 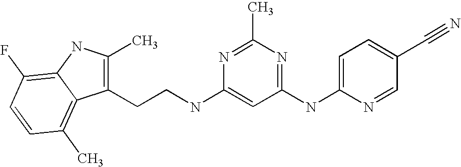 | 0.83 | 415.468 | 416 |
| 166 | 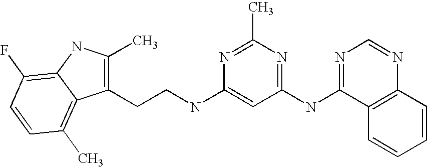 | 0.83 | 441.506 | 442 |
| 167 | 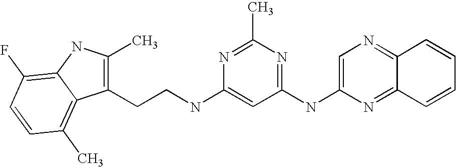 | 0.9 | 441.506 | 442 |
| 168 | 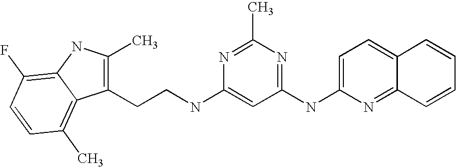 | 1.0 | 440.518 | 441 |
| 167 | 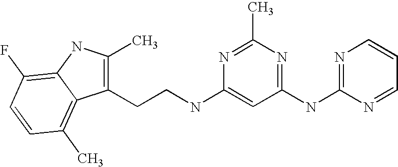 | 0.77 | 391.447 | 392 |
| 168 | 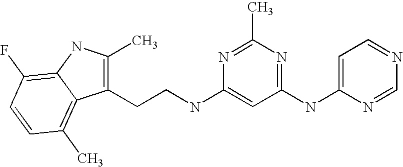 | 0.9 | 391.447 | 392 |

### BIOLOGICAL EXAMPLES

### 1. Detection of the Antagonism of the Human Prostaglandin E₂ (Subtype EP2 Receptor Signal

### 1.1 Principle of Detection

The binding of PGE₂ to the EP₂ subtype of the human PGE₂ receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labelled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nM). The two signals are measured in a suitable measuring instrument with a time lag, i.e. after the background fluorescence has declined. Any increase in the low FRET signal caused by prostaglandin E₂ addition (measured as well ratio change=emission_{665 nm}/emission₆₂₀ nm*10 000) shows the effect of antagonists.

### 1.2. Detection Method

1.2.1 Antagonism Assay (Data for Each Well of a 384-Well plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # 1-7018), and then 15 μl thereof are transferred into a media-free cell culture plate which has been washed with KRSB shortly beforehand.

After preincubation at room temperature (RT) for 30 minutes, 5 μl of a 4×PGE₂ solution (11 nM) are added, and incubation is carried out in the presence of the agonist at RT for a further 60 min (volume: 20 μl) before the reaction is then stopped by adding 5 μl of lysis buffer and incubated at RT for a further 20 min (volume: ˜25 μl). The cell lysate is then transferred into a measuring plate and measured in accordance with the manufacturer's information (cyclic AMP kit Cisbio International # 62AMPPEC).

### 1.2.2 Agonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # 1-7018), and then 15 μl thereof are transferred into a media-free cell culture plate which has been washed with KRSB shortly beforehand.

After incubation at room temperature (RT; volume: 15 μl) for 60 minutes, the reaction is then stopped by adding 5 μl of lysis buffer and incubated at RT for a further 20 min (volume: 20 μl). The cell lysate is then transferred into a measuring plate and measured in accordance with the manufacturer's information (cyclic AMP kit Cisbio International # 62AMPPEC).

### 2. The EP₂ Subtype of the PGE₂ Receptor and the Preovulatory Cumulus Expansion

### 2.1. Background:

In the preovulatory antral follicle, the oocyte is surrounded by cumulus cells which form a dense ring of cells around the oocyte. After the LH peak (lutenizing hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important component of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18):10501-6.) show a markedly reduced cumulus expansion and severe subfertility, demonstrating the importance of the prostanoid EP₂ receptor for this process.

### 2.2 Cumulus Expansion Assay In Vitro

Folliculogenesis is induced in immature female mice at an age of 14-18 days by a single dose (intraperitoneal) of 5-101. U. of PMSG (Pregnant Mare Serum Gonadotropine; Sigma G-4877, Lot 68H0909). 47-50 hours after the injection, the ovaries are removed and the cumulus-oocyte complexes are removed. The cumulus complex is not yet expanded at this stage.

The cumulus-oocyte complexes are then incubated with prostaglandin E₂ (PGE₂) (0.3 μM), vehicle control (ethanol) or test substances for 20-24 hours.

Medium: alpha-MEM medium with 0.1 mM IBMX, pyruvates (0.23 mM) glutamines (2 mM), pen/strep 100 IU/ml pen. and 100 μg/ml strep.) and HSA (8 mg/ml)). Cumulus expansion is then established through the division into four stages (according to Vanderhyden et al. Dev Biol. 1990 August; 140(2):307-317).

TABLE 1
Example of the biological activity of the compounds of the invention
(measured by the cAMP antagonism assay):

| Substance of Example | Antagonism [IC₅₀, μM] |
|---|---|
| 6 | 1.6 |
| 17 | 1.4 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07075499.9, filed Jun. 21, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of the formula I 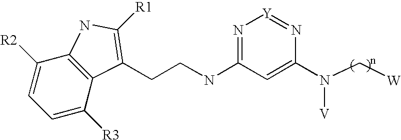
where
Y is a CH group or a C(C₁-C₄-alkyl) group,
V is a hydrogen, a C₁-C₄-alkyl group,
n is 0, 1 or 2,
W is a 6-10-membered, mono- or bicyclic aryl ring which is in each case unsubstituted or optionally substituted once to three times,
a 5-10-membered, mono- or bicyclic heteroaryl ring which is in each case unsubstituted or optionally substituted once to three times,
an 8-12-membered aryl- or heteroaryl-cycloalkyl or -cycloalkenyl group which is in each case unsubstituted or optionally substituted once to three times,
an 8-12-membered aryl- or heteroaryl-heterocyclyl or -heterocyclenyl group which is in each case unsubstituted or optionally substituted once to three times,
a 3-12-membered, mono-, bi- or tricyclic cycloalkyl radical which is in each case unsubstituted or optionally substituted once, where the substituents are linked either directly or via a spacer U to W and may be selected from the group of halogen, cyano, R⁴, OR⁴, OC(O)R⁴, S(O)ₙR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴,C(O)NR⁴R⁵,
or
in the case where n=0 together with V is a pyrrolidine, piperidine, morpholine or thiomorpholine residue which is in each case unsubstituted or optionally substituted once,
or else
in the case where n=0 together with V is a piperazine residue, which is unsubstituted or optionally N-substituted, where the substituents are linked either directly or via a spacer U to W and may be selected from the group of halogen, cyano, R⁴, OR⁴, OC(O)R⁴, S(O)NR⁴ where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
U is a C₁-C₄-alkylene, C₂-C₄-alkenylidene, C₂-C₄-alkynylidene, O—C₁-C₄-alkylene, C(O)—C₁-C₄-alkylene, S(O)ₙ—C₁-C₄-alkylene, where n is 0, 1, 2, N(R⁵)—C₁-C₄-alkylene, C(O)—N(R⁵)—C₁-C₄-alkylene, N(R⁵)—C(O)—C₁-C₄-alkylene spacer,
R¹ is a C₁-C₄-alkyl group or cyano,
R² is a hydrogen, halogen, cyano, a C₁-C₄-alkyl group,
R³ is a hydrogen, halogen, cyano, a C₁-C₄-alkyl group,
R⁴ is a hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or heteroaryl group, where the aryl radical is 6-membered and the heteroaryl radical is 5 or 6-membered,
R⁵ is a hydrogen, a C₁-C₄-alkyl group and
R⁴, R⁵ together form a 3-6-membered cycloalkyl or a heteroatom-containing ring,
and the isomers, diastereomers, enantiomers and salts thereof, and cyclodextrin clathrates.

2. A compound as claimed in claim 1, where
where
Y is a CH group or a C(C₁-C₄-alkyl) group,
V is a hydrogen, a CH₃ group,
n is 0, 1 or 2,
W is a 6-10-membered, mono- or bicyclic aryl ring which is in each case unsubstituted or optionally substituted once to three times,
a 5-10-membered, mono- or bicyclic heteroaryl ring which is in each case unsubstituted or optionally substituted once to three times,
an 8-12-membered aryl- or heteroaryl-cycloalkyl or -cycloalkenyl group which is in each case unsubstituted or optionally substituted once to three times,
an 8-12-membered aryl- or heteroaryl-heterocyclyl or -heterocyclenyl group which is in each case unsubstituted or optionally substituted once to three times,
a 3-6 membered cycloalkyl radical which is in each case unsubstituted or optionally substituted once, where the substituents are linked either directly or via a spacer U to W and may be selected from the group of halogen, cyano, R⁴, OR⁴, OC(O)R⁴, S(O)NR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
or
in the case where n=0 together with V is a pyrrolidine, piperidine, morpholine or thiomorpholine residue which is in each case unsubstituted or optionally substituted once,
or else
in the case where n=0 together with V is a piperazine radical, which is unsubstituted or optionally N-substituted, where the substituents are linked either directly or via a spacer U to W and may be selected from the group of halogen, cyano, R⁴, OR⁴, OC(O)R⁴, S(O)NR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
U is a C₁-C₄-alkylene, C₂-C₄-alkenylidene, C₂-C₄-alkynylidene, O—C₁-C₄-alkylene, C(O)—C₁-C₄-alkylene, S(O)ₙ—C₁-C₄-alkylene, where n is 0, 1, 2, N(R⁵)—C₁-C₄-alkylene, C(O)—N(R⁵)—C₁-C₄-alkylene, N(R⁵)—C(O)—C₁-C₄-alkylene spacer,
R¹ is a C₁-C₄-alkyl group or cyano,
R² is a hydrogen, halogen, cyano, a C₁-C₄-alkyl group,
R³ is a hydrogen, halogen, cyano, a C₁-C₄-alkyl group,
R⁴ is a hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or heteroaryl group, where the aryl radical is 6-membered and the heteroaryl radical is 5 or 6-membered,
R⁵ is a hydrogen, a C₁-C₄-alkyl group,
R⁴, R⁵ together form a 3-6-membered cycloalkyl or a heteroatom-containing ring.

3. A compound as claimed in claim 1, where
Y is a CH group or a C(C₁-alkyl) group,
V is a hydrogen, a CH₃ group,
n is 0, 1 or 2,
W is a 6-10-membered, mono- or bicyclic aryl ring which is in each case unsubstituted or optionally substituted once to three times,
a 5-10-membered, mono- or bicyclic heteroaryl ring which is in each case unsubstituted or optionally substituted once to three times,
an 8-12-membered aryl- or heteroaryl-cycloalkyl or -cycloalkenyl group which is in each case unsubstituted or optionally substituted once to three times,
an 8-12-membered aryl- or heteroaryl-heterocyclyl or -heterocyclenyl group which is in each case unsubstituted or optionally substituted once to three times,
a 3-6-membered cycloalkyl radical which is in each case unsubstituted or optionally substituted once, where the substituents are linked either directly or via a spacer U to W and may be selected from the group of halogen, cyano, R⁴, OR⁴, OC(O)R⁴, S(O)ₙR⁴ where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
or
in the case where n=0 together with V is a pyrrolidine, piperidine, morpholine or thiomorpholine residue which is in each case unsubstituted or optionally substituted once,
or else
in the case where n=0 together with V is a piperazine residue which is unsubstituted or optionally N-substituted, where the substituents are linked either directly or via a spacer U to W and may be selected from the group of halogen, cyano, R⁴, OR⁴, OC(O)R⁴, S(O)NR⁴, where n is 0, 1, 2, SO₂NR⁴R⁵, SO₂NR⁵C(O)R⁴, NR⁴R⁵, NR⁵C(O)R⁴, NR⁵SO₂R⁴, C(O)NR⁵SO₂R⁴, C(OH)R⁴R⁵, C(O)R⁴, C(NOH)R⁴, CO₂R⁴, C(O)NR⁴R⁵,
U is a C₁-C₄-alkylene, C₂-C₄-alkenylidene, C₂-C₄-alkynylidene, O—C₁-C₄-alkylene, C(O)—C₁-C₄-alkylene, S(O)ₙ—C₁-C₄-alkylene, where n is 0, 1, 2, N(R⁵)—C₁-C₄-alkylene, C(O)—N(R⁵)—C₁-C₄-alkylene, N(R⁵)—C(O)—C₁-C₄-alkylene spacer,
R¹ is a Ct-alkyl group or cyano,
R² is a hydrogen, halogen, cyano, a C₁-alkyl group,
R³ is a hydrogen, halogen, cyano, a C₁-alkyl group,
R⁴ is a hydrogen, a C₁-C₄-alkyl group, a C₂-C₄-alkenyl group, a C₂-C₄-alkynyl group, a C₃-C₆-cycloalkyl group, a CH₂—C₃-C₆-cycloalkyl group, a 6-membered aryl ring, a 5-6-membered heteroaryl ring or a CH₂-aryl or heteroaryl group, where the aryl radical is 6-membered and the heteroaryl radical is 5 or 6-membered,
R⁵ is a hydrogen, a C₁-C₄-alkyl group and
R⁴, R⁵ together form a 3-6-membered cycloalkyl or a heteroatom-containing ring.

4. A compound according to claim 1, wherein said compound is:
1. N-[2-(7-Fluoro-2,4-dimethyl-1H-indol-3-yl)ethyl]-N′-pyridin-2-ylpyrimidine-4,6-diamine
2. N-[2-(7-Fluoro-2,4-dimethyl-1H-indol-3-yl)ethyl]-N′-pyridin-3-ylpyrimidine-4,6-diamine
3. N-(3-Chlorophenyl)-N′-[2-(7-fluoro-2,4-dimethyl-1H-indol-3-yl)ethyl]-pyrimidine-4,6-diamine
4. N-[2-(7-Fluoro-2,4-dimethyl-1H-indol-3-yl)ethyl]-N′-(3-trifluoromethylphenyl)-pyrimidine-4,6-diamine
5. N-[2-(7-Fluoro-2,4-dimethyl-1H-indol-3-yl)ethyl]-N′-(4-fluorophenyl)-pyrimidine-4,6-diamine
6. N-[2-(7-Fluoro-2,4-dimethyl-1H-indol-3-yl)ethyl]-N′-pyridin-3-ylmethylpyrimidine-4,6-diamine
7. N-[2-(7-Fluoro-2,4-dimethyl-1H-indol-3-yl)ethyl]-N′-phenylpyrimidine-4,6-diamine
8. N-[2-(7-Fluoro-2,4-dimethyl-1H-indol-3-yl)ethyl]-N′-(2-methoxyphenyl)-pyrimidine-4,6-diamine
9. N-[2-(7-Fluoro-2,4-dimethyl-1H-indol-3-yl)ethyl]-N′-(3-methoxyphenyl)-pyrimidine-4,6-diamine
10. N-[2-(7-Fluoro-2,4-dimethyl-1H-indol-3-yl)ethyl]-N′-(4-methoxyphenyl)-pyrimidine-4,6-diamine
11. N-(4-Chlorophenyl)-N′-[2-(7-fluoro-2,4-dimethyl-1H-indol-3-yl)ethyl]-pyrimidine-4,6-diamine
12. N-Cyclohexyl-N′-[2-(7-fluoro-2,4-dimethyl-1H-indol-3-yl)ethyl]pyrimidine-4,6-diamine
13. N-(4-Dimethylaminophenyl)-N′-[2-(7-fluoro-2,4-dimethyl-1H-indol-3-yl)ethyl]-pyrimidine-4,6-diamine
14. N-[2-(7-Fluoro-2,4-dimethyl-1H-indol-3-yl)ethyl]-N′-pyrazin-2-ylpyrimidine-4,6-diamine
15. N-Benzyl-N′-[2-(7-fluoro-2,4-dimethyl-1H-indol-3-yl)ethyl]pyrimidine-4,6-diamine
16. N-[2-(7-Fluoro-2,4-dimethyl-1H-indol-3-yl)ethyl]-N′-(4-methoxybenzyl)-pyrimidine-4,6-diamine
17. N-Biphenyl-2-ylmethyl-N′-[2-(7-fluoro-2,4-dimethyl-1H-indol-3-yl)ethyl]-pyrimidine-4,6-diamine
18. N-[2-(7-Fluoro-2,4-dimethyl-1H-indol-3-yl)ethyl]-N′-(4-[1,2,4]triazol-1-yl-phenyl)pyrimidine-4,6-diamine
19. [2-(7-Fluoro-2,4-dimethyl-1H-indol-3-yl)ethyl]-[6-(2,3,5,6-tetrahydro-[1,2′]bipyrazinyl-4-yl)pyrimidin-4-yl]amine
20. N-[2-(7-Fluoro-2,4-dimethyl-1H-indol-3-yl)ethyl]-N′-(4-methylbenzyl)-pyrimidine-4,6-diamine
21. N-[2-(7-Fluoro-2,4-dimethyl-1H-indol-3-yl)ethyl]-N′-(4-trifluoromethylphenyl)-pyrimidine-4,6-diamine
22. N-Biphenyl-3-ylmethyl-N′-[2-(7-fluoro-2,4-dimethyl-1H-indol-3-yl)ethyl]-pyrimidine-4,6-diamine
23. 4-{6-[2-(7-Fluoro-2,4-dimethyl-1H-indol-3-yl)ethylamino]pyrimidin-4-ylamino}-N-thiazol-2-ylbenzenesulfonamide
24. N-(4,6-Dimethylpyrimidin-2-yl)-4-{6-[2-(7-fluoro-2,4-dimethyl-1H-indol-3-yl)ethylamino]pyrimidin-4-ylamino}benzenesulfonamide
25. N-[2-(7-Fluoro-2,4-dimethyl-1H-indol-3-yl)ethyl]-N′-(6-methylpyridin-2-yl)-pyrimidine-4,6-diamine
26. 5-{6-[2-(7-Fluoro-2,4-dimethyl-1H-indol-3-yl)ethylamino]pyrimidin-4-ylamino}-indan-1-one
27. 6-{6-[2-(7-Fluoro-2,4-dimethyl-1H-indol-3-yl)ethylamino]pyrimidin-4-ylamino}-3,4-dihydro-2H-naphthalen-1-one
28. 5-{6-[2-(7-Fluoro-2,4-dimethyl-1H-indol-3-yl)ethylamino]pyrimidin-4-ylamino}-isoindole-1,3-dione
29. 6-{6-[2-(7-Fluoro-2,4-dimethyl-1H-indol-3-yl)ethylamino]pyrimidin-4-ylamino}-nicotinamide
30. N-[2-(7-Fluoro-2,4-dimethyl-1H-indol-3-yl)ethyl]-N′-naphthalen-1-yl-pyrimidine-4,6-diamine
31. N-Benzo[1,3]dioxol-5-yl-N′-[2-(7-fluoro-2,4-dimethyl-1H-indol-3-yl)ethyl]-pyrimidine-4,6-diamine
32. N-[2-(7-Fluoro-2,4-dimethyl-1H-indol-3-yl)ethyl]-N′-(1H-indol-5-yl)-pyrimidine-4,6-diamine
33. N-(1H-Benzotriazol-5-yl)-N′-[2-(7-fluoro-2,4-dimethyl-1H-indol-3-yl)ethyl]-pyrimidine-4,6-diamine
34. N-[2-(7-Fluoro-2,4-dimethyl-1H-indol-3-yl)ethyl]-N′-indan-5-ylpyrimidine-4,6-diamine
35. 4-{6-[2-(7-Fluoro-2,4-dimethyl-1H-indol-3-yl)ethylamino]pyrimidin-4-ylamino}-isoindole-1,3-dione
36. 4-{6-[2-(7-Fluoro-2,4-dimethyl-1H-indol-3-yl)ethylamino]pyrimidin-4-ylamino}-benzamide
37. 6-{6-[2-(7-Fluoro-2,4-dimethyl-1H-indol-3-yl)ethylamino]pyrimidin-4-ylamino}-2,3-dihydrophthalazine-1,4-dione
38. N-[2-(7-Fluoro-2,4-dimethyl-1H-indol-3-yl)ethyl]-N′-(5-methyl-2H-pyrazol-3-yl)pyrimidine-4,6-diamine
39. N-[2-(7-Fluoro-2,4-dimethyl-1H-indol-3-yl)ethyl]-N′-quinolin-3-ylpyrimidine-4,6-diamine
40. N-[2-(7-Fluoro-2,4-dimethyl-1H-indol-3-yl)ethyl]-N′-quinolin-5-ylpyrimidine-4,6-diamine
41. N-[2-(7-Fluoro-2,4-dimethyl-1H-indol-3-yl)ethyl]-N′-quinolin-8-ylpyrimidine-4,6-diamine
42. 5-{6-[2-(7-Fluoro-2,4-dimethyl-1H-indol-3-yl)ethylamino]pyrimidin-4-ylamino}-2-methylisoindole-1,3-dione
43. N-[2-(7-Fluoro-2,4-dimethyl-1H-indol-3-yl)ethyl]-N′-(1H-pyrazolo[3,4-d]pyrimidin-4-yl)pyrimidine-4,6-diamine
44. N-(2,5-Dimethyl-2H-pyrazol-3-yl)-N′-[2-(7-fluoro-2,4-dimethyl-1H-indol-3-yl)ethyl]pyrimidine-4,6-diamine
45. N-[2-(7-Fluoro-2,4-dimethyl-1H-indol-3-yl)ethyl]-N′-(2-trifluoromethyl-1H-benzoimidazol-5-yl)pyrimidine-4,6-diamine
46. N-[2-(7-Fluoro-2,4-dimethyl-1H-indol-3-yl)ethyl]-N′-[3-(1H-tetrazol-5-yl)-phenyl]pyrimidine-4,6-diamine
47. 3-{6-[2-(7-Fluoro-2,4-dimethyl-1H-indol-3-yl)ethylamino]pyrimidin-4-ylamino}-benzenesulfonamide
48. N-[2-(7-Fluoro-2,4-dimethyl-1H-indol-3-yl)ethyl]-N′-(1H-indazol-5-yl)-pyrimidine-4,6-diamine
49. N-[2-(7-Fluoro-2,4-dimethyl-1H-indol-3-yl)ethyl]-N′-(1H-indazol-6-yl)-pyrimidine-4,6-diamine
50. N-[2-(7-Fluoro-2,4-dimethyl-1H-indol-3-yl)ethyl]-N′-isoquinolin-1-yl-pyrimidine-4,6-diamine
51. N-Benzothiazol-6-yl-N′-[2-(7-fluoro-2,4-dimethyl-1H-indol-3-yl)ethyl]-pyrimidine-4,6-diamine
52. N-(4-tert-Butylphenyl)-N′-[2-(7-fluoro-2,4-dimethyl-1H-indol-3-yl)ethyl]-pyrimidine-4,6-diamine
53. N-[2-(7-Fluoro-2,4-dimethyl-1H-indol-3-yl)ethyl]-N′-(5-trifluoromethylpyridin-2-yl)pyrimidine-4,6-diamine
54. N-[2-(7-Fluoro-2,4-dimethyl-1H-indol-3-yl)ethyl]-N′-isoquinolin-3-yl-pyrimidine-4,6-diamine
55. (4-{6-[2-(7-Fluoro-2,4-dimethyl-1H-indol-3-yl)ethylamino]pyrimidin-4-ylamino}phenyl)acetonitrile
56. N-[2-(7-Fluoro-2,4-dimethyl-1H-indol-3-yl)ethyl]-N′-(2,4,5,6-tetrahydro-cyclopentapyrazol-3-yl)pyrimidine-4,6-diamine
57. N-(2,3-Dihydrobenzo[1,4]dioxin-6-yl)-N′-[2-(7-fluoro-2,4-dimethyl-1H-indol-3-yl)ethyl]pyrimidine-4,6-diamine
58. N-[2-(7-Fluoro-2,4-dimethyl-1H-indol-3-yl)ethyl]-N′-(4-phenoxyphenyl)-pyrimidine-4,6-diamine
59. 7-{6-[2-(7-Fluoro-2,4-dimethyl-1H-indol-3-yl)ethylamino]pyrimidin-4-ylamino}-4-methylchromen-2-one
60. N-[2-(7-Fluoro-2,4-dimethyl-1H-indol-3-yl)ethyl]-N′-(2-methylbenzothiazol-5-yl)pyrimidine-4,6-diamine
61. [2-(7-Fluoro-2,4-dimethyl-1H-indol-3-yl)ethyl](2-methyl-6-piperidin-1-yl-pyrimidin-4-yl)amine
62. N-Biphenyl-4-yl-N′-[2-(7-fluoro-2,4-dimethyl-1H-indol-3-yl)ethyl]-2-methylpyrimidine-4,6-diamine
63. N-[2-(7-Fluoro-2,4-dimethyl-1H-indol-3-yl)ethyl]-2-methyl-N′-pyridin-2-yl-pyrimidine-4,6-diamine
64. N-[2-(7-Fluoro-2,4-dimethyl-1H-indol-3-yl)ethyl]-2-methyl-N′-pyridin-3-yl-pyrimidine-4,6-diamine
65. N-(3-Chlorophenyl)-N′-[2-(7-fluoro-2,4-dimethyl-1H-indol-3-yl)ethyl]-2-methylpyrimidine-4,6-diamine
66. N-[2-(7-Fluoro-2,4-dimethyl-1H-indol-3-yl)ethyl]-2-methyl-N′-(3-trifluoromethylphenyl)pyrimidine-4,6-diamine
67. N-[2-(7-Fluoro-2,4-dimethyl-1H-indol-3-yl)ethyl]-N′-(4-fluorophenyl)-2-methylpyrimidine-4,6-diamine
68. N-[2-(7-Fluoro-2,4-dimethyl-1H-indol-3-yl)ethyl]-N′-(2-fluorophenyl)-2-methylpyrimidine-4,6-diamine
69. N-[2-(7-Fluoro-2,4-dimethyl-1H-indol-3-yl)ethyl]-2-methyl-N′-(2-trifluoromethylphenyl)pyrimidine-4,6-diamine
70. N-[2-(7-Fluoro-2,4-dimethyl-1H-indol-3-yl)ethyl]-2-methyl-N′-pyridin-4-yl-pyrimidine-4,6-diamine
71. N-[2-(7-Fluoro-2,4-dimethyl-1H-indol-3-yl)ethyl]-2-methyl-N′-phenethyl-pyrimidine-4,6-diamine
72. N-[2-(7-Fluoro-2,4-dimethyl-1H-indol-3-yl)ethyl]-2-methyl-N′-pyridin-2-ylmethylpyrimidine-4,6-diamine
73. N-[2-(7-Fluoro-2,4-dimethyl-1H-indol-3-yl)ethyl]-2-methyl-N′-pyridin-3-ylmethylpyrimidine-4,6-diamine
74. N-[2-(7-Fluoro-2,4-dimethyl-1H-indol-3-yl)ethyl]-2-methyl-N′-pyridin-4-ylmethylpyrimidine-4,6-diamine
75. N-[2-(7-Fluoro-2,4-dimethyl-1H-indol-3-yl)ethyl]-2-methyl-N′-phenyl-pyrimidine-4,6-diamine
76. N-[2-(7-Fluoro-2,4-dimethyl-1H-indol-3-yl)ethyl]-N′-(2-methoxyphenyl)-2-methylpyrimidine-4,6-diamine
77. N-[2-(7-Fluoro-2,4-dimethyl-1H-indol-3-yl)ethyl]-N′-(3-methoxyphenyl)-2-methylpyrimidine-4,6-diamine
78. N-[2-(7-Fluoro-2,4-dimethyl-1H-indol-3-yl)ethyl]-N′-(4-methoxyphenyl)-2-methylpyrimidine-4,6-diamine
79. N-(4-Chlorophenyl)-N′-[2-(7-fluoro-2,4-dimethyl-1H-indol-3-yl)ethyl]-2-methylpyrimidine-4,6-diamine
80. N-Cyclohexyl-N′-[2-(7-fluoro-2,4-dimethyl-1H-indol-3-yl)ethyl]-2-methylpyrimidine-4,6-diamine
81. N-(4-Dimethylaminophenyl)-N′-[2-(7-fluoro-2,4-dimethyl-1H-indol-3-yl)ethyl]-2-methylpyrimidine-4,6-diamine
82. N-[2-(7-Fluoro-2,4-dimethyl-1H-indol-3-yl)ethyl]-2-methyl-N′-pyrazin-2-yl-pyrimidine-4,6-diamine
83. N-Benzyl-N′-[2-(7-fluoro-2,4-dimethyl-1H-indol-3-yl)ethyl]-2-methylpyrmidine-4,6-diamine
84. N-[2-(7-Fluoro-2,4-dimethyl-1H-indol-3-yl)ethyl]-N′-(4-methoxybenzyl)-2-methylpyrimidine-4,6-diamine
85. N-[2-(7-Fluoro-2,4-dimethyl-1H-indol-3-yl)ethyl]-2-methyl-N′-(3-methyl-isothiazol-5-yl)pyrimidine-4,6-diamine
86. [2-(7-Fluoro-2,4-dimethyl-1H-indol-3-yl)ethyl]-[2-methyl-6-(4-pyridin-2-yl-piperazin-1-yl)pyrimidin-4-yl]amine
87. N-Biphenyl-2-ylmethyl-N′-[2-(7-fluoro-2,4-dimethyl-1H-indol-3-yl)ethyl]-2-methylpyrimidine-4,6-diamine
88. [2-(7-Fluoro-2,4-dimethyl-1H-indol-3-yl)ethyl]-[2-methyl-6-(4-pyrimidin-2-yl-piperazin-1-yl)pyrimidin-4-yl]amine
89. [6-(4-Benzylpiperazin-1-yl)-2-methylpyrimidin-4-yl][2-(7-fluoro-2,4-dimethyl-1H-indol-3-yl)ethyl]amine
90. N-[2-(7-Fluoro-2,4-dimethyl-1H-indol-3-yl)ethyl]-2-methyl-N′-(4-[1,2,4]triazol-1-ylphenyl)pyrimidine-4,6-diamine
91. N-(4-{6-[2-(7-Fluoro-2,4-dimethyl-1H-indol-3-yl)ethylamino]-2-methylpyrimidin-4-ylamino}phenyl)acetamide
92. N-(2-Fluorobenzyl)-N′-[2-(7-fluoro-2,4-dimethyl-1H-indol-3-yl)ethyl]-2-methylpyrimidine-4,6-diamine
93. N-Cyclohexylmethyl-N′-[2-(7-fluoro-2,4-dimethyl-1H-indol-3-yl)ethyl]-2-methylpyrimidine-4,6-diamine
94. N-(4-Fluorobenzyl)-N′-[2-(7-fluoro-2,4-dimethyl-1H-indol-3-yl)ethyl]-2-methylpyrimidine-4,6-diamine
95. N-[2-(7-Fluoro-2,4-dimethyl-1H-indol-3-yl)ethyl]-2-methyl-N′-(3-trifluoromethylbenzyl)pyrimidine-4,6-diamine
96. N-[2-(7-Fluoro-2,4-dimethyl-1H-indol-3-yl)ethyl]-2-methyl-N′-(4-methyl-benzyl)pyrimidine-4,6-diamine
97. N-[2-(7-Fluoro-2,4-dimethyl-1H-indol-3-yl)ethyl]-2-methyl-N′-(4-trifluoromethylbenzyl)pyrimidine-4,6-diamine
98. N-[2-(7-Fluoro-2,4-dimethyl-1H-indol-3-yl)ethyl]-2-methyl-N′-(4-trifluoromethylphenyl)pyrimidine-4,6-diamine
99. N-Biphenyl-4-ylmethyl-N′-[2-(7-fluoro-2,4-dimethyl-1H-indol-3-yl)ethyl]-2-methylpyrimidine-4,6-diamine
100. N-Biphenyl-3-ylmethyl-N′-[2-(7-fluoro-2,4-dimethyl-1H-indol-3-yl)ethyl]-2-methylpyrimidine-4,6-diamine
101. 4-{6-[2-(7-Fluoro-2,4-dimethyl-1H-indol-3-yl)ethylamino]-2-methylpyrimidin-4-ylamino}-N-methylbenzamide
102. 4-{6-[2-(7-Fluoro-2,4-dimethyl-1H-indol-3-yl)ethylamino]-2-methylpyrimidin-4-ylamino}-N-thiazol-2-ylbenzenesulfonamide
103. 4-{6-[2-(7-Fluoro-2,4-dimethyl-1H-indol-3-yl)ethylamino]-2-methylpyrimidin-4-ylamino}-N-pyrimidin-2-ylbenzenesulfonamide
104. N-(4,6-Dimethylpyrimidin-2-yl)-4-{6-[2-(7-fluoro-2,4-dimethyl-1H-indol-3-yl)ethylamino]-2-methylpyrimidin-4-ylamino}benzenesulfonamide
105. N-Acetyl-4-{6-[2-(7-fluoro-2,4-dimethyl-1H-indol-3-yl)ethylamino]-2-methylpyrimidin-4-ylamino}benzenesulfonamide
106. N-[2-(7-Fluoro-2,4-dimethyl-1H-indol-3-yl)ethyl]-2-methyl-N′-(6-methylpyridin-2-yl)pyrimidine-4,6-diamine
107. 5-{6-[2-(7-Fluoro-2,4-dimethyl-1H-indol-3-yl)ethylamino]-2-methylpyrimidin-4-ylamino}indan-1-one
108. 6-{6-[2-(7-Fluoro-2,4-dimethyl-1H-indol-3-yl)ethylamino]-2-methylpyrimidin-4-ylamino}-3,4-dihydro-2H-naphthalen-1-one
109. 5-{6-[2-(7-Fluoro-2,4-dimethyl-1H-indol-3-yl)ethylamino]-2-methylpyrimidin-4-ylamino} isoindole-1,3-dione
110. 6-{6-[2-(7-Fluoro-2,4-dimethyl-1H-indol-3-yl)ethylamino]-2-methylpyrimidin-4-ylamino}nicotinamide
111. N-[2-(7-Fluoro-2,4-dimethyl-1H-indol-3-yl)ethyl]-2-methyl-N′-naphthalen-1-ylpyrimidine-4,6-diamine
112. N-Benzo[1,3]dioxol-5-yl-N′-[2-(7-fluoro-2,4-dimethyl-1H-indol-3-yl)ethyl]-2-methylpyrimidine-4,6-diamine
113. N-[2-(7-Fluoro-2,4-dimethyl-1H-indol-3-yl)ethyl]-N′-(1H-indol-5-yl)-2-methylpyrimidine-4,6-diamine
114. N-[2-(7-Fluoro-2,4-dimethyl-1H-indol-3-yl)ethyl]-N′-indan-5-yl-2-methylpyrimidine-4,6-diamine
115. 4-{6-[2-(7-Fluoro-2,4-dimethyl-1H-indol-3-yl)ethylamino]-2-methylpyrimidin-4-ylamino} isoindole-1,3-dione
116. 4-{6-[2-(7-Fluoro-2,4-dimethyl-1H-indol-3-yl)ethylamino]-2-methylpyrimidin-4-ylamino}benzamide
117. 6-{6-[2-(7-Fluoro-2,4-dimethyl-1H-indol-3-yl)ethylamino]-2-methylpyrimidin-4-ylamino}-2,3-dihydrophthalazine-1,4-dione
118. N-[2-(7-Fluoro-2,4-dimethyl-1H-indol-3-yl)ethyl]-2-methyl-N′-(5-methyl-2H-pyrazol-3-yl)pyrimidine-4,6-diamine
119. N-[2-(7-Fluoro-2,4-dimethyl-1H-indol-3-yl)ethyl]-2-methyl-N′-quinolin-3-yl-pyrimidine-4,6-diamine
120. N-[2-(7-Fluoro-2,4-dimethyl-1H-indol-3-yl)ethyl]-2-methyl-N′-quinolin-5-yl-pyrimidine-4,6-diamine
121. N-[2-(7-Fluoro-2,4-dimethyl-1H-indol-3-yl)ethyl]-2-methyl-N′-quinolin-6-yl-pyrimidine-4,6-diamine
122. N-[2-(7-Fluoro-2,4-dimethyl-1H-indol-3-yl)ethyl]-2-methyl-N′-quinolin-8-yl-pyrimidine-4,6-diamine
123. 5-{6-[2-(7-Fluoro-2,4-dimethyl-1H-indol-3-yl)ethylamino]-2-methylpyrimidin-4-ylamino}-2-methylisoindole-1,3-dione
124. N-[2-(7-Fluoro-2,4-dimethyl-1H-indol-3-yl)ethyl]-2-methyl-N′-(1H-pyrazolo[3,4-d]pyrimidin-4-yl)pyrimidine-4,6-diamine
125. N-(2,5-Dimethyl-2H-pyrazol-3-yl)-N′-[2-(7-fluoro-2,4-dimethyl-1H-indol-3-yl)ethyl]-2-methylpyrimidine-4,6-diamine
126. N-[2-(7-Fluoro-2,4-dimethyl-1H-indol-3-yl)ethyl]-2-methyl-N′-(2-trifluoromethyl-1H-benzoimidazol-5-yl)pyrimidine-4,6-diamine
127. N-[2-(7-Fluoro-2,4-dimethyl-1H-indol-3-yl)ethyl]-2-methyl-N′-[3-(1H-tetrazol-5-yl)phenyl]pyrimidine-4,6-diamine
128. N-[2-(7-Fluoro-2,4-dimethyl-1H-indol-3-yl)ethyl]-N′-(6-methoxypyridin-3-yl)-2-methylpyrimidine-4,6-diamine
129. N-[2-(7-Fluoro-2,4-dimethyl-1H-indol-3-yl)ethyl]-2-methyl-N′-[1,3,5]triazin-2-ylpyrimidine-4,6-diamine
130. 3-{6-[2-(7-Fluoro-2,4-dimethyl-1H-indol-3-yl)ethylamino]-2-methylpyrimidin-4-ylamino}benzenesulfonamide
131. N-[2-(7-Fluoro-2,4-dimethyl-1H-indol-3-yl)ethyl]-N′-(1H-indazol-5-yl)-2-methylpyrimidine-4,6-diamine
132. N-[2-(7-Fluoro-2,4-dimethyl-1H-indol-3-yl)ethyl]-N′-(1H-indazol-6-yl)-2-methylpyrimidine-4,6-diamine
133. N-[2-(7-Fluoro-2,4-dimethyl-1H-indol-3-yl)ethyl]-N′-isoquinolin-1-yl-2-methylpyrimidine-4,6-diamine
134. N-Benzothiazol-6-yl-N′-[2-(7-fluoro-2,4-dimethyl-1H-indol-3-yl)ethyl]-2-methylpyrimidine-4,6-diamine
135. N-[2-(7-Fluoro-2,4-dimethyl-1H-indol-3-yl)ethyl]-2-methyl-N′-[1,2,4]triazin-3-ylpyrimidine-4,6-diamine
136. N-(4-tert-Butylphenyl)-N′-[2-(7-fluoro-2,4-dimethyl-1H-indol-3-yl)ethyl]-2-methylpyrimidine-4,6-diamine
137. N-[2-(7-Fluoro-2,4-dimethyl-1H-indol-3-yl)ethyl]-2-methyl-N′-(5-trifluoromethylpyridin-2-yl)pyrimidine-4,6-diamine
138. N-[2-(7-Fluoro-2,4-dimethyl-1H-indol-3-yl)ethyl]-N′-isoquinolin-3-yl-2-methylpyrimidine-4,6-diamine
139. N-[2-(7-Fluoro-2,4-dimethyl-1H-indol-3-yl)ethyl]-2-methyl-N′-(2,4,5,6-tetrahydrocyclopentapyrazol-3-yl)pyrimidine-4,6-diamine
140. N-(2,3-Dihydrobenzo[1,4]dioxin-6-yl)-N′-[2-(7-fluoro-2,4-dimethyl-1H-indol-3-yl)ethyl]-2-methylpyrimidine-4,6-diamine
141. N-[2-(7-Fluoro-2,4-dimethyl-1H-indol-3-yl)ethyl]-2-methyl-N′-(4-phenoxy-phenyl)pyrimidine-4,6-diamine
142. 7-{6-[2-(7-Fluoro-2,4-dimethyl-1H-indol-3-yl)ethylamino]-2-methylpyrimidin-4-ylamino}-4-methylchromen-2-one or
143. N-[2-(7-Fluoro-2,4-dimethyl-1H-indol-3-yl)ethyl]-2-methyl-N′-(2-methyl-benzothiazol-5-yl)pyrimidine-4,6-diamine.

5. A medicament comprising at least one of the compounds according to claim 1.

6. A medicament according to claim 5, further comprising one or more suitable formulating substances and/or carriers.

7. A method for the treatment and prophylaxis of disorders, comprising administering to a patient in need thereof a medicament according to claim 5.

8. A method for the treatment and prophylaxis of disorders connected with the EP₂ receptor, comprising administering to a patient in need thereof a medicament according to claim 5.

9. A method for the treatment and prophylaxis of fertility impairments, comprising administering to a patient in need thereof a medicament according to claim 5.

10. A method for the treatment and prophylaxis of painful menstruation, comprising administering to a patient in need thereof a medicament according to claim 5.

11. A method for the treatment and prophylaxis of endometriosis, comprising administering to a patient in need thereof a medicament according to claim 5.

12. A method for modulating the EP₂ receptor, comprising administering to a patient in need thereof a medicament according to claim 5.

13. A method for the treatment and prophylaxis of pain, comprising administering to a patient in need thereof a medicament according to claim 5.

14. A method for controlling fertility/contraception, comprising administering to a patient in need thereof a medicament according to claim 6.

15. A method for the treatment and prophylaxis of osteoporosis, comprising administering to a patient in need thereof a medicament according to claim 5.

16. A method for the treatment and prophylaxis of cancer, comprising administering to a patient in need thereof a medicament according to claim 5.

17. A method for the treatment and prophylaxis of inflammatory disorders such as, for example, Crohn's disease, comprising administering to a patient in need thereof a medicament according to claim 5.

18. A pharmaceutical composition comprising a compound according to claim 1, wherein said composition is suitable for enteral, parenteral, vaginal and oral administration.
